(19) United States
(12) Patent Application Publication
(10) Pub. No.: US 2011/0130374 A1
(43) Pub. Date: Jun. 2, 2011

(54) Small Pyrimidine Derivatives and Methods of Use Thereof

(75) Inventors: Omri EREZ, Rehovot (IL); Philippe NAKACHE, Nes Ziona (IL)

(73) Assignee: BIOSEARCH(2007) LTD., Jerusalem (IL)

(21) Appl. No.: 12/956,744

(22) Filed: Nov. 30, 2010

Related U.S. Application Data
(60) Provisional application No. 61/265,085, filed on Nov. 30, 2009.

(51) Int. Cl. A61K 31/573; A61K 31/506; A61P 29/00; A61P 37/08; A61P 19/02; A61P 11/06; A61P 37/06; A61P 1/04; A61P 17/06; A61P 35/00; A61P 35/04

(52) U.S. Cl. 514/171; 514/275

(57) ABSTRACT

Provided are pyrimidine derivatives which are ubiquitination inhibitors that inhibit the ubiquitin ligase activity, particularly of POSH polypeptides, and are useful for the treatment of cancer, angiogenesis disorders, and inflammatory disorders.

### FIELD

The presently described subject matter relates to small pyrimidine derivatives, which are inhibitors of the ubiquitin ligase activity of a human polypeptide, particularly to POSH inhibitors, and to compositions and methods for treatment of cell migration related conditions, disorders or diseases.

### BACKGROUND

Potential drug target validation involves determining whether a DNA, RNA or protein molecule is implicated in a disease process and is therefore a suitable target for development of new therapeutic drugs. Drug discovery, the process by which bioactive compounds are identified and characterized, is a critical step in the development of new treatments for human diseases. The landscape of drug discovery has changed dramatically due to the genomics revolution. DNA and protein sequences are yielding a host of new drug targets and an enormous amount of associated information.

The identification of genes and proteins involved in various disease states or key biological processes, such as inflammation and immune response, is a vital part of the drug design process. Many diseases and disorders could be treated or prevented by decreasing the expression of one or more genes involved in the molecular etiology of the condition if the appropriate molecular target is identified and appropriate antagonists developed. For example, cancer, in which one or more cellular oncogenes become activated and result in the unchecked progression of cell cycle processes, could be treated by antagonizing appropriate cell cycle control genes. Furthermore many human genetic diseases, such as Huntington's disease, and certain prion conditions, which are influenced by both genetic and epigenetic factors, result from the inappropriate activity of a polypeptide as opposed to the complete loss of its function. Accordingly, antagonizing the aberrant function of such mutant genes would provide a means of treatment. Drug therapy strategies for treating such diseases and disorders have frequently employed molecular antagonists which target the polypeptide product of the disease gene(s). However, the discovery of relevant gene or protein targets is often difficult and time consuming.

Targeted proteins undergoing selective degradation, presumably through the actions of a ubiquitin-dependent proteosome, are covalently tagged with ubiquitin through the formation of an isopeptide bond between the C-terminal glycyl residue of ubiquitin and a specific lysyl residue in the substrate protein. This process is catalyzed by a ubiquitin-activating enzyme (E1) and a ubiquitin-conjugating enzyme (E2), and in some instances may also require auxiliary substrate recognition proteins (E3s). Following the linkage of the first ubiquitin chain, additional molecules of ubiquitin may be attached to lysine side chains of the previously conjugated moiety to form branched multi-ubiquitin chains.

The conjugation of ubiquitin to protein substrates is a multi-step process. In an initial ATP requiring step, a thioester is formed between the C-terminus of ubiquitin and an internal cysteine residue of an E1 enzyme. Activated ubiquitin may then be transferred to a specific cysteine on one of several E2 enzymes. Finally, these E2 enzymes donate ubiquitin to protein substrates, typically with the assistance of a E3 protein, also known as a ubiquitin ligase enzyme. In certain instances, substrates are recognized directly by the ubiquitin-conjugated E2 enzyme. Ubiquitin (ub) protein ligases (E3's) are functionally defined as proteins that facilitate the covalent linkage (conjugation) of one or multiple ubiquitin molecules to a substrate protein in the presence of E1 (ub-activating enzyme) and an E2 (ub carrier protein). In the absence of a protein substrate, E3's can catalyze self-ubiquitination, that is, transfer of activated ubiquitin from E2 to a lysine residue acceptor site on the E3 polypeptide, a reaction termed self-ubiquitination. Similar to trans ubiquitination, self-ubiquitination is dependent on the presence of E1, E2 and an intact E3 functional module i.e. RING finger or HECT domain (Lorick K L et al., Proc Natl Acad Sci USA. 1999 96:11364-9; Kao W H et al., J Virol. 2000 74:6408-6417).

It is also known that the ubiquitin system plays a role in a wide range of cellular processes including intracellular transport, cell cycle progression, apoptosis, and turnover of many membrane receptors. In viral infections, the ubiquitin system is involved not only with assembly, budding and release, but also with repression of host proteins such as p53, which may lead to a viral-induced neoplasm. The HIV Vpu protein interacts with an E3 protein that regulates IκB degradation, and is thought to promote apoptosis of infected cells by indirectly inhibiting NF-κB activity (Bour et al. (2001) J Exp Med 194:1299-311; U.S. Pat. No. 5,932,425). The ubiquitin system regulates protein function by both monoubiquitination and polyubiquitination. Polyubiquitination is primarily associated with protein degradation.

Cell migration is crucial for various biological processes, including embryonic development, wound healing and immune responses. Aberrant cell migration is also a common feature to many pathological conditions such as cancer cell invasion, angiogenesis and metastasis (Chiang and Massague, 2008), inflammation diseases (Mackay, 2008), retinopathy such as age-related macular degeneration (AMD) (Das and McGuire, 2003) and others.

### SUMMARY

It was found, according to the presently described subject matter, that certain pyrimidine derivatives act as ubiquitination inhibitors, and by inhibiting POSH protein-mediated ubiquitination, are capable of blocking cell migration.

The presently described subject matter is directed to small-molecules which strongly inhibit cell migration. The present subject matter further describes the potential of inhibitory molecules as therapeutic agents for different indications involving pathological cell migration such as cancer, different inflammation-related indications, and angiogenesis-related indications such as age-related macular degeneration (AMD), retinopathies and others.

In an embodiment, the presently described subject matter is directed to a method for treating a cell migration disease, disorder or condition in a subject, or a disease, disorder or condition associated with cell migration, comprising administering to the subject a therapeutically effective amount of a presently described compound.

In another embodiment, the presently described subject matter is directed to a method for treating a cell migration disease, disorder or condition in a subject, or a disease, disorder or condition associated with cell migration, comprising administering to the subject a therapeutically effective amount of a pharmaceutical composition comprising a presently described compound of general formula I.

The presently described subject matter is further directed to the use of a small molecule, which is a pyrimidine derivative of the general formula I depicted hereinafter for the treatment of a disease, disorders, or conditions related to or associated with cell migration. In some embodiments, the presently described subject matter relates to the use of the compounds of formula I, which are the compounds herein designated compounds 1, 2, 3, 4, 5, 6 and 7 for the treatment of diseases, disorders or conditions related to or associated with cell migration, i.e., a cell migration disease, disorder or condition.

In another embodiment, the presently described subject matter is directed to a method for treating a cell migration disease, disorder or condition in a subject, or a disease, disorder or condition associated with cell migration, wherein the cell migration disease, disorder or condition is cancer, an inflammatory condition, or an angiogensis related condition.

### BRIEF DESCRIPTION OF THE FIGURES

FIG. 1A is a graphical representation showing the inhibitory effect of compound 1 and compound 2 on cell migration.

FIG. 1B is a graphical representation showing the inhibitory effect of compound 1 and compound 2 on cell migration.

FIG. 1C is a graphical representation showing the effect of compound 1 on soft agar colony formation (MDA-MB231 cells).

FIG. 1D is a graphical representation showing the effect of compound 1 on soft agar colony formation (A375 cells).

FIG. 1E is a graphical representation showing the inhibitory effect of compound 3 on cell migration.

FIG. 1F is a graphical representation showing the effect of compound 3 on soft agar colony formation (MDA-MB231 cells).

FIG. 2 is a photographic representation of the inhibitory effect of compound 1 on cell migration of various cell lines.

FIG. 3 is a photographic representation of the inhibitory effect of compound 1 on HUVEC cells tube-formation.

FIG. 4A is a graphical representation showing efficacy of compound 1 in inhibiting lung metastases in female B6D2F1 mice implanted with B16F10 murine melanoma cells.

FIG. 4B is a graphical representation showing efficacy of compound 3 in inhibiting lung metastases in female B6D2F1 mice implanted with B16F10 murine melanoma cells.

FIG. 5 is a graphical representation showing that compounds 1 and 3 have the same anti-inflammatory activity as the control.

### DETAILED DESCRIPTION

Definitions

Without limiting the scope to further possible definitions, as used in the present specification, the terms hereinbelow are defined as follows:

The term “hydrocarbyl” means a radical derived from a hydrocarbon that may be acyclic or cyclic, saturated, unsaturated or aromatic, hydrocarbyl radical, of 1-20 carbon atoms, of 1 to 10, of 1 to 6, or of 2-3 carbon atoms, and includes alkyl, alkenyl, alkynyl, cycloalkyl, cycloalkenyl, aralkyl and aryl.

The “alkyl,” “alkenyl,” or “alkynyl” radical is a “C₁-C₁₀ alkyl” or a “C₁-C₄ alkyl,” a “C₂-C₁₀ alkenyl” or a “C₂-C ₄ alkenyl,” a “C₂-C₁₀ alkynyl” or a “C₂-C₄ alkynyl,” respectively, that may be straight or branched and may be interrupted by one or more heteroatoms selected from O, S and/or N, and/or substituted by one or more radicals selected from the group consisting of halogen, aryl, heterocyclyl, heteroaryl, nitro, epoxy, epithio, —OR, —SR, —COR, —COOR—NRR′, —CONRR′, —NRCOR′—SO₃R, —SO₂R, —SO₂NRR′ and —NRSO₂R, wherein R and R′, are each independently, H, hydrocarbyl or heterocyclyl, or R and R′ together with the nitrogen atom to which they are attached form a saturated 5-7 membered heterocyclic ring, optionally containing 1 or 2 further heteroatoms selected from N, S and/or O, and wherein said further N atom is optionally substituted by hydrocarbyl.

The term “lower alkyl,” refers to a “C₁-C₄ alkyl” that may be straight or branched alkyl radical having 1-4 carbon atoms and may be interrupted by one or more heteroatoms selected from O, S and/or N, and/or substituted as defined above. Lower alkyls include for example methyl, ethyl, n-propyl, isopropyl, n-butyl, isobutyl, tert-butyl. In one embodiment the lower alkyl is methyl.

Any “C₂-C₄ alkenyl” is a straight or branched unsaturated radical having 2-4 carbon atoms and one or two double bonds, e.g. an alkadienyl radical, wherein the alkenyl radical can optionally have a terminal double bond, and includes for example vinyl, prop-2-en-1-yl, but-3-en-1-yl. Any “C₂-C₄ alkynyl” is a straight or branched unsaturated radical having 2-4 carbon atoms and one or more triple bonds and includes, for example, ethynyl, propynyl, butynyl. All alkyl, alkenyl, and alkynyl radicals can be substituted as defined herein.

The term “carbocyclyl” herein includes the terms “cycloalkyl” and “cycloalkenyl,” which refer to a “C₅-C₆ cycloalkyl” or “C₅-C₆ cycloalkenyl,” respectively, namely, 5-6 completely saturated or partially unsaturated carbocyclic groups and include cyclopentyl, cyclohexyl, cyclopentenyl and cyclohexenyl, that may be substituted by one or more radicals selected from the group consisting of halogen, hydrocarbyl, heterocyclyl, nitro, epoxy, epithio, OR, —SR, —COR, —COOR—NRR′, —CONRR′, —NRCOR′—SO₃R, —SO₂R, —SO₂NRR′ and —NRSO₂R, wherein R and R′, are each independently, H, hydrocarbyl or heterocyclyl, or R′ and R″ together with the nitrogen atom to which they are attached form a saturated heterocyclic ring, optionally containing 1 or 2 further heteroatoms selected from N, S and/or O, and wherein the further N atom is optionally substituted by hydrocarbyl.

The term “aryl” refers to a “C₆-C₁₄” aromatic carbocyclic group having 6 to 14 carbon atoms or 6 to 10 carbon atoms, consisting of a single, bicyclic or tricyclyc ring, such as phenyl, naphthyl and antracenyl, that may be substituted by one or more radicals as defined herein above.

The term “heterocyclyl” means a radical derived from saturated or partially unsaturated (non-aromatic) monocyclic, bicyclic or tricyclic heterocycle, of 3 to 12, 5 to 10, or 5 to 6, ring members, of which ring members one to three is a heteroatom selected from O, S and/or N. Non-limiting examples of non-aromatic heterocyclyl include dihydrofuryl, tetrahydrofuryl, dihydrothienyl, pyrrolydinyl, pyrrolynyl, dihydropyridyl, piperidinyl, piperazinyl, morpholino, 1,3-dioxanyl, and the like. The heterocyclyl radical may be substituted by one or more radicals as defined herein above. It is to be understood that when a polycyclic heterocyclyl ring is substituted, the substitutions may be in any of the carbocyclic and/or heterocyclic rings.

The term “heteroaryl” as used herein, means a radical derived from a mono- or poly-cyclic heteroaromatic ring containing one to three heteroatoms selected from the group consisting of O, S and N. Particular examples are pyrrolyl, furyl, thienyl, pyrazolyl, imidazolyl, oxazolyl, thiazolyl, pyridyl, quinolinyl, isoquinolinyl, pyridazinyl, pyrimidinyl, pyrazinyl, 1,3,4-triazinyl, 1,2,3-triazinyl, 1,3,5-triazinyl, benzofuryl, isobenzofuryl, indolyl, imidazo[1,2-a]pyridyl, benzimidazolyl, benzthiazolyl and benzoxazolyl, benzodiazepinyl, and other radicals derived from further polycyclic heteroaromatic rings. The heteroaryl radical may be substituted by one or more radicals as defined herein above. It is to be understood that when a polycyclic heteroaryl ring is substituted, the substitutions may be in any of the carbocyclic and/or heterocyclic rings. In one embodiment the heteroaryl is thienyl.

The term “halogen” refers to fluoro, chloro, bromo or iodo. In some embodiments, the halogen is chloro.

The groups —NR₇R₈ or —NRR′ or may be —NH₂, when R₇ (or R) and R₈ (or R′) are both hydrogen, or secondary amino when R₇ (or R) is H and R₈ (or R′) is C₁-C₄ alkyl, or tertiary amino when R₇ (or R) and R₈ (or R′) are each C₁-C₄ alkyl, or R₇ or R₈ (R and R′, respectively) together with the nitrogen atom to which they are attached may form a saturated, for example a 5- or 6-membered, heterocyclic ring, optionally containing 1 or 2 further heteroatoms selected from nitrogen, oxygen and/or sulfur. Such rings may be substituted by lower alkyl, aralkyl, haloalkyl or hydroxyalkyl, for example at a further N atom. Examples of such rings include, without being limited to, pyrrolidino, piperidino, morpholino, thiomorpholino, piperazino, N-alkylpiperazino, e.g. N-methylpiperazino, and diazepino.

Any alkoxy, alkylthio or alkanoyl groups formed by the radicals OR₆ (or OR), SR₆, (or SR), COR₆ (or COR), when R₆ (or R) is alkyl, are C₁-C₄ alkoxy, C₁-C₄ alkylthio and C₂-C₄ alkanoyl groups, respectively. Examples of alkoxy are methoxy, ethoxy, propyloxy, butoxy, and the like, and examples of alkylthio are the same but replacing the —O— by —S—, and examples of alkanoyl are acetyl, propanoyl, butanoyl, and the like. All alkoxy, thioalkyl, and alkanoyl radicals may be substituted as defined above. In one embodiment, the C₁-C₄ alkoxy is methoxy.

As used herein, the terms “administering,” “administration,” and like terms refer to any method which, in sound medical practice, delivers a composition to a subject in such a manner as to provide a therapeutic effect. One aspect of the present subject matter provides for oral administration of a therapeutically effective amount of a composition of the present subject matter to a patient in need thereof. Other suitable routes of administration can include parenteral, subcutaneous, intravenous, intramuscular, intraperitoneal, transdermal, buccal, intrathecal, intracranial, intranasal or topical routes. Alternatively, or concurrently, administration may be by the oral route. The dosage administered will be dependent upon the age, health, and weight of the recipient, kind of concurrent treatment, if any, frequency of treatment, and the nature of the effect desired.

The phrase “angiogenesis-related indications” means any angiogenesis-related condition where aberrant cell migration is a feature. Such angiogenesis-related conditions can include conditions related to excessive or undesired angiogenesis such as diabetic blindness; chronic inflammation; arthritis; age-related macular degeneration; retinopathy; rheumatoid arthritis; osteoarthritis; Crohn's disease; psoriasis; cancer; Alzheimer's disease; restenosis; pulmonary fibrosis; asthma; angiofibroma; neovascular glaucoma; arteriovenous malformations; nonunion fractures; lupus and other connective tissue disorders; Osler-Weber syndrome; atherosclerotic plaques; corneal graft neovascularization; Pyogenic granuloma; retrolental fibroplasias; scleroderma; granulations, hemangioma; trachoma; hemophilic joints; peritoneal endometriosis; adiposity; and vascular adhesions.

The phrase “angiogenesis inhibitor” means any substance that inhibits angiogenesis. Suitable angiogenesis inhibitors include, but are not limited to, bevacizumab (AVASTIN™), sunitinib (SUTENT™), sorafenib (NEXAVAR™), thalidomide (THALOMID™), lenalidomide (REVLIMID™), panitumumab (VECTIBIX™), cetuximab (ERBITUX™), and erlotinib (TARCEVA™).

The phrase “anti-cancer agent” means any substance that is effective for treating cancer. Such substances can include, for example, kinase inhibitors, for example one or more tyrosine kinase inhibitors. Suitable tyrosine kinase inhibitors include, but are not limited to, imatinib, dasatinib, axitinib, bosutinib, cediranib, erlotinib, gefitinib, lapatinib, lestaurtinib, nilotinib, semaxanib, sutinib, toceranib, vandetanib, and vatalanib.

The phrase “anti-inflammatory agent” means any substance that reduces or surpresses inflammation. Suitable anti-inflammatory agents include, but are not limited to, corticosteroid including for example cortisol, aldosterone, hydrocortisone, hydrocortisone acetate, cortisone acetate, tixocortol pivalate, prednisolone, methylprednisolone, prednisone, triamcinolone acetonide, triamcinolone alcohol, mometasone, amcinonide, budesonide, desonide, flucinonide, fluocinolone acetonide, halcinonide, betamethasone, betamethasone sodium phosphate, dexamethasone, dexamethasone dodium phosphate, flucortolone, hydrocortisone-17-butyrate, hydrocortisone-17-valerate, aclometasone dipropionate, betamethasone valerate, betamethasone dipropionate, prednicarbate, clobetasone-17-butyrate, clobetasol-17-propionate, flucortolone caproate, fluocortolone pivalate, and fluprednidene acetate; a non-steroidal anti-inflammatory drug including for example, a cox-2 inhibitor, nimesulide, diclofenac, licofelone, aspirin, ibuprofen, and naproxen; immune selective anti-inflammatory derivatives such as phenylalanine-glutamine-glycine and its D-isomeric form; and herbs including for example, Harpagophytum, hyssop, ginger, turmeric, Arnica montana, willow bark, and cannabis.

The term “cancer” is the generic name of a group of diseases that are characterised by abnormal, uncontrolled cell division, the ability to invade normal tissues and the ability to spread to other parts of the body. Such cancers can include anal cancer; astrocytoma; leukemia; lymphoma; head and neck cancer; liver cancer; testicular cancer; cervical cancer; sarcoma; hemangioma; esophageal cancer; eye cancer; laryngeal cancer; mouth cancer; mesothelioma; myeloma; oral cancer; rectal cancer; throat cancer; bladder cancer; breast cancer; uterine cancer; ovarian cancer; prostate cancer; lung cancer; colon cancer; pancreatic cancer; renal cell carcinoma; gastric cancer; skin cancer; including basal cell carcinoma, melanoma, and squamous cell carcinoma; oral squamous cell carcinoma; colorectal cancer; glioblastoma multiforme; endometrial cancer; and malignant glioma.

The phrase “cancer chemotherapeutic agent” means any known chemotherapeutic agent that may be used for combination therapy with the presently described subject matter, and includes, but is not limited to alkylating agents, including mechlorethamine, cyclophosphamide, ifosfamide, melphalan, chlorambucil, dicarbazine, streptazocine, carmustine, lomustine, semustine, chlorozotocin, busulfan, triethylenemelamine, thiotepa, hexamethylmelamine; antimetabolites, including methotrexate; fluorouracil; 5-fluorouracil; floxuridine (5′-fluoro-2′-deoxyuridine); idoxuridine; cytarabine; N-phosphonoacetyl-L-aspartate; 5-azacytidine; azaribine; 6-azauridine; pyrazofuran; 3-deazauridine; acivicin; purine analogs, including thioguanine, mercaptopurine, azathioprine, pentostatin, erythrohydroxynonyladenine; vinca alkaloids, including vincristine and vinblastine; epipodophyllotoxins, including etoposide and teniposide; antibiotics, including dactinomycin, daunorubicin, doxorubicin, bleomycin sulfate, plicamycin, mitomycin; enzymes, including L-asparaginase; platinum coordination complexes, including cisplatin, carboplatin; hydroxyurea, procarbazine, mitotane; and hormones or related agents, including adrenocorticosteroids such as prednisone and prednisolone; aminoglutethimide; progestins such as hydroxyprogesterone caproate, medroxyprogesterone acetate, megesterol acetate, estrogens and androgens such as diethylstilbestrol, fluoxymesterone, ethynyl estradiol, antiestrogens such as tamoxifen, and gonadotropin-releasing hormone analogs such as leuprolide.

In practicing the presently described methods for treating cancer, the present compounds may be administered together with at least one known chemotherapeutic agent and/or at least one anti-cancer agent as part of a unitary pharmaceutical composition. Alternatively, the present compounds may be administered apart from at least one known cancer chemotherapeutic agent and/or at least one anti-cancer agent. In one embodiment, the present compounds and at least one known cancer chemotherapeutic agent and/or at least one anti-cancer agent are administered substantially simultaneously, i.e. the compounds are administered at the same time or one after the other, so long as the compounds reach therapeutic levels in the blood at the same time. In another embodiment, the present compounds and at least one known cancer chemotherapeutic agent and/or at least one anti-cancer agent are administered according to their individual dose schedule, so long as the compounds reach therapeutic levels in the blood.

Similarly, another embodiment of the presently described subject matter is directed to a method of treating cancer by administering the presently described compounds in combination with radiation therapy. In this embodiment, the presently described compounds may be administered at the same time as the radiation therapy is administered or at a different time.

The phrase “disease, disorder or condition related to cell migration” means any disease, disorder or condition where aberrant cell migration is a feature. Such diseases, disorders or conditions can include cancer, different inflammation-related indications, and angiogenesis-related indications such as age-related macular degeneration (AMD), retinopathies and others.

As used herein, the term “carrier,” “excipient,” or “adjuvant” refers to any component of a pharmaceutical composition that is not the drug substance.

As used herein, the phrases “drug product,” “pharmaceutical dosage form,” “final dosage form,” and the like, refer to the combination of one or more drug substances and one or more excipients (i.e., pharmaceutical composition) that is administered to a patient in need of treatment, and can be in the form of a solution, an aqueous solution, an emulsion, a suspension, tablets, capsules, patches, suppositories, a cream, a gel, a lotion, and the like.

Pharmaceutical compositions containing a presently described compound or its pharmaceutically acceptable salts as the active ingredient can be prepared according to conventional pharmaceutical compounding techniques. See, for example, Remington's Pharmaceutical Sciences, 18th Ed. (1990, Mack Publishing Co., Easton, Pa.).

The phrase “inflammation-related indications” means any inflammatory condition where aberrant cell migration is a feature. Such inflammatory conditions can include pulmonary fibrosis; ischaemic heart disease; autoimmune diseases such as Crohn's disease, dermatomyositis, diabetes mellitus, Guillain-Barre syndrome, hashimoto's disease, idiopathic thrombocytopenic purpura, mixed connective tissue disease, myasthenia gravis, narcolepsy, pemphigus vulgaris, pernicious anaemia, polymyositis, primary biliary cirrhosis, Sjogren's syndrome, temporal arteritis, ulcerative colitis, vasculitis, Wegener's granulomatosis, systemic lupus erythematosus, lupus nephritis, Goodpasture's syndrome, haemolytic anaemia, thyrotoxicosis, multiple sclerosis, and scleroderma; chronic inflammatory conditions such as asthma, rheumatoid arthritis, osteoarthritis, septicaemia, artherosclerosis, chronic renal disease, inflammatory bowel disease, and vasculitis; peritonitis; ocular inflammatory diseases such as giant papillary conjunctivitis, uveitis, and seasonal allergic conjunctivitis; chronic prostatitis; glomerulonephritis; hypersensitivities; inflammatory bowel diseases; pelvic inflammatory disease; reperfusion injury; transplant rejection; Chediak-Higashi syndrome; chronic granulomatous disease; urinary tract inflammatory conditions; interstitial cystitis; ulcerative colitis; systemic sclerosis; dermatomyositis; polymyositis, and inclusion body myositis.

As used herein, the term “pharmaceutically acceptable carrier” refers to a non-toxic, inert solid, semi-solid liquid filler, diluent, encapsulating material, formulation auxiliary of any type, or simply a sterile aqueous medium, such as saline. Some examples of the materials that can serve as pharmaceutically acceptable carriers are sugars, such as lactose, glucose and sucrose, starches such as corn starch and potato starch, cellulose and its derivatives such as sodium carboxymethyl cellulose, ethyl cellulose and cellulose acetate; powdered tragacanth; malt, gelatin, talc; excipients such as cocoa butter and suppository waxes; oils such as peanut oil, cottonseed oil, safflower oil, sesame oil, olive oil, corn oil and soybean oil; glycols, such as propylene glycol, polyols such as glycerin, sorbitol, mannitol and polyethylene glycol; esters such as ethyl oleate and ethyl laurate, agar; buffering agents such as magnesium hydroxide and aluminum hydroxide; alginic acid; pyrogen-free water; isotonic saline, Ringer's solution; ethyl alcohol and phosphate buffer solutions, as well as other non-toxic compatible substances used in pharmaceutical formulations.

Some non-limiting examples of substances which can serve as a carrier herein include sugar, starch, cellulose and its derivatives, powered tragacanth, malt, gelatin, talc, stearic acid, magnesium stearate, calcium sulfate, vegetable oils, polyols, alginic acid, pyrogen-free water, isotonic saline, phosphate buffer solutions, cocoa butter (suppository base), emulsifier as well as other non-toxic pharmaceutically compatible substances used in other pharmaceutical formulations. Wetting agents and lubricants such as sodium lauryl sulfate, as well as coloring agents, flavoring agents, excipients, tabletting agents, stabilizers, antioxidants, and preservatives may also be present. Any non-toxic, inert, and effective carrier may be used to formulate the compositions contemplated herein. Suitable pharmaceutically acceptable carriers, excipients, and diluents in this regard are well known to those of skill in the art, such as those described in The Merck Index, Thirteenth Edition, Budavari et al., Eds., Merck & Co., Inc., Rahway, N.J. (2001); the CTFA (Cosmetic, Toiletry, and Fragrance Association) International Cosmetic Ingredient Dictionary and Handbook, Tenth Edition (2004); and the “Inactive Ingredient Guide”, U.S. Food and Drug Administration (FDA) Center for Drug Evaluation and Research (CDER) Office of Management, the contents of all of which are hereby incorporated by reference in their entirety. Examples of pharmaceutically acceptable excipients, carriers and diluents useful in the present compositions include distilled water, physiological saline, Ringer's solution, dextrose solution, Hank's solution, and DMSO.

These additional inactive components, as well as effective formulations and administration procedures, are well known in the art and are described in standard textbooks, such as Goodman and Gillman's: The Pharmacological Bases of Therapeutics, 8th Ed., Gilman et al. Eds. Pergamon Press (1990) and Remington's Pharmaceutical Sciences, 18th Ed., Mack Publishing Co., Easton, Pa. (1990), both of which are incorporated by reference herein in their entirety.

The carrier may comprise, in total, from about 0.1% to about 99.99999% by weight of the pharmaceutical compositions presented herein.

The presently described pharmaceutical compositions can be in the form of oral compositions. The oral compositions contemplated herein may take the form of tablets, capsules, soft-gels, hard gels, solutions, suspensions, powders, dispersible granules, cachets, combinations thereof, or any other oral pharmaceutical dosage form as would commonly be known in the art.

A solid carrier can be one or more substances which may also act as diluents, flavoring agents, solubilizers, lubricants, suspending agents, binders, or tablet disintegration agents; it can also be an encapsulating material. In powders, the carrier can be a finely divided solid which is in admixture with the active compound. In a tablet, the active compound can be mixed with a carrier having the necessary binding properties in suitable proportions and compacted in the size and shape desired. Non-limiting examples of suitable solid carriers include magnesium carbonate, magnesium stearate, talc, cornstarch, sugar, lactose, pectin, dextrin, starch, gelatin, tragacanth, methyl cellulose, sodium carboxymethylcellulose, hydroxypropyl methylcellulose, hydroxyethylcellulose, hydroxypropylcellulose, other cellulose derivatives, a low melting wax, cocoa butter, and the like.

As used herein, the phrase “pharmaceutically acceptable salts” refers to salts of certain ingredient(s) which possess the same activity as the unmodified compound(s) and which are neither biologically nor otherwise undesirable. A salt can be formed with, for example, organic or inorganic acids. Non-limiting examples of suitable acids include acetic acid, acetylsalicylic acid, adipic acid, alginic acid, ascorbic acid, aspartic acid, benzoic acid, benzenesulfonic acid, bisulfic acid, boric acid, butyric acid, camphoric acid, camphorsulfonic acid, carbonic acid, citric acid, cyclopentanepropionic acid, digluconic acid, dodecylsulfic acid, ethanesulfonic acid, formic acid, fumaric acid, glyceric acid, glycerophosphoric acid, glycine, glucoheptanoic acid, gluconic acid, glutamic acid, glutaric acid, glycolic acid, hemisulfic acid, heptanoic acid, hexanoic acid, hippuric acid, hydrobromic acid, hydrochloric acid, hydroiodic acid, hydroxyethanesulfonic acid, lactic acid, maleic acid, malic acid, malonic acid, mandelic acid, methanesulfonic acid, mucic acid, naphthylanesulfonic acid, naphthylic acid, nicotinic acid, nitrous acid, oxalic acid, pelargonic, phosphoric acid, propionic acid, saccharin, salicylic acid, sorbic acid, succinic acid, sulfuric acid, tartaric acid, thiocyanic acid, thioglycolic acid, thiosulfuric acid, tosylic acid, undecylenic acid, and naturally and synthetically derived amino acids.

If organic bases are used, poorly volatile bases are preferably employed, for example low molecular weight alkanolamines such as ethanolamine, diethanolamine, N-ethylethanolamine, N-methyldiethanolamine, triethanolamine, diethylaminoethanol, 2-amino-2-methyl-n-propanol, dimethylaminopropanol, 2-amino-2-methylpropanediol, and triisopropanolamine Ethanolamine is suitable in this regard. Further poorly volatile bases which may be mentioned are, for example, ethylenediamine, hexamethylenediamine, morpholine, piperidine, piperazine, cyclohexylamine, tributylamine, dodecylamine, N,N-dimethyldodecylamine, stearylamine, oleylamine, benzylamine, dibenzylamine, N-ethylbenzylamine, dimethylstearylamine, N-methylmorpholine, N-methylpiperazine, 4-methylcyclohexylamine, and N-hydroxyethylmorpholine.

Salts of quaternary ammonium hydroxides such as trimethylbenzylammonium hydroxide, tetramethylammonium hydroxide, or tetraethylammonium hydroxide can also be used, as can guanidine and its derivatives, in particular its alkylation products. However, it is also possible to employ as salt-forming agents, for example, low molecular weight alkylamines such as methylamine, ethylamine, or triethylamine. Suitable salts for the components to be employed according to the present subject matter are also those with inorganic cations, for example alkali metal salts, in particular sodium, potassium, or ammonium salts, alkaline earth metal salts such as, in particular, the magnesium or calcium salts, as well as salts with bi- or tetravalent cations, for example the zinc, aluminum, or zirconium salts. Also contemplated are salts with organic bases, such as dicyclohexylamine salts; methyl-D-glucamine; and salts with amino acids, such as arginine, lysine, and so forth. Also, the basic nitrogen-containing groups can be quaternized with such agents as lower alkyl halides, such as methyl, ethyl, propyl, and butyl chlorides, bromides, and iodides; dialkyl sulfates, such as dimethyl, diethyl, dibutyl, and diamyl sulfates; long chain halides, such as decyl, lauryl, myristyl, and stearyl chlorides, bromides, and iodides; asthma halides, such as benzyl and phenethyl bromides; and others. Aqueous or oil-soluble or dispersible products are thereby obtained.

As used herein, the terms “POSH,” “POSH protein(s)” or “POSH polypeptide(s)” are used interchangeably and refer to a polypeptide that includes in its amino acid sequence a RING domain and at least one SH3 domain. In some instances, the POSH protein may have 3 or 4 SH3 domains.

The terms “POSH-mediated ubiquitination” or “POSH protein-mediated ubiquitination” are used interchangeably and refer to any ubiquitination process that requires the involvement of a POSH protein.

POSH intersects with and regulates a wide range of key cellular functions that may be manipulated by affecting the level of and/or activity of POSH polypeptides or POSH-AP polypeptides. Many features of POSH, and particularly human POSH, are described in PCT patent publications WO03/095971A2 and WO03/078601A2, the teachings of which are incorporated by reference herein in their entirety.

As described in the above-referenced publications, native human POSH is a large polypeptide containing a RING domain and four SH3 domains. POSH is a ubiquitin ligase (also termed an “E3” enzyme); the RING domain mediates ubiquitination of, for example, the POSH polypeptide itself POSH interacts with a large number of proteins and participates in a host of different biological processes. As demonstrated in this disclosure, POSH associates with a number of different proteins in the cell. POSH co-localizes with proteins that are known to be located in the trans-Golgi network, implying that POSH participates in the trafficking of proteins in the secretory system. The term “secretory system” should be understood as referring to the membrane compartments and associated proteins and other molecules that are involved in the movement of proteins from the site of translation to a location within a vacuole, a compartment in the secretory pathway itself, a lysosome or endosome or to a location at the plasma membrane or outside the cell. Commonly cited examples of compartments in the secretory system include the endoplasmic reticulum, the Golgi apparatus and the cis and trans Golgi networks.

In addition, POSH interactor Rac1 is elevated in various cancers and is involved in cell migration and invasion (Bosco, Mulloy et al. 2009). Rac is a therapeutic target for BCR-ABL tyrosine kinase-induced myeloproliferative disease such as chronic myelogenous leukemia (CML) (Thomas, Cancelas et al. 2007; Thomas, Cancelas et al. 2008). CML patients are treated with tyrosine kinase inhibitors such as Imatinib (Gleevec) that target p210-BCR-ABL, the constitutively active tyrosine kinase. The interaction of POSH and Rac1 raises the possibility that combination therapy of tyrosine kinase inhibitors, including but not limited to Imatinib, Dasatinib, Axitinib, Bosutinib, Cediranib, Erlotinib, Gefitinib, Lapatinib, Lestaurtinib, Nilotinib, Semaxanib, Sunitinib, Toceranib, Vandetanib, and Vatalanib, and a POSH inhibitor will significantly improved long-term hematologic remissions. Moreover, recently it was demonstrated that angiogenesis inhibitors targeting the VEGF pathway demonstrate antitumor effects in various models but concomitantly elicit tumor adaptation and progression to stages of greater malignancy, mainly by increasing invasiveness and lymphatic and distant metastasis (Paez-Ribes, Allen et al. 2009). In addition, VEGFR/PDGFR kinase inhibitor Sunitinib/SU11248 can accelerate metastatic tumor growth and decrease overall survival in mice receiving short-term therapy in various metastasis assays (Ebos, Lee et al. 2009). Taken together, the physical and functional interaction of POSH with Rac1 and the inhibitory effect of POSH inhibitors on cell migration and invasion suggest that combination therapy of POSH inhibitor with tyrosine kinase inhibitors such as Imatinib, Dasatinib and others and angiogenesis inhibitors targeting VEGFR/PDGFR pathways such as Sunitinib, anti-VEGFR blocking antibodies and others can improve the therapy clinical outcome of various type of cancers and prevents therapy induced metastasis.

The terms “ubiquitination inhibitor,” “POSH inhibitor” or “POSH protein inhibitor” are used interchangeably and refer to a pyrimidine derivative of formula I herein that inhibits a POSH activity as defined in PCT/US02/36366 (WO 03/095972), hereby incorporated herein by reference in its entirety as if fully disclosed herein, including POSH protein-mediated ubiquitination.

The active agent is preferably administered in a therapeutically effective amount. As used herein, the term “safe and effective amount” refers to the quantity of a component which is sufficient to yield a desired therapeutic response without undue adverse side effects (such as toxicity, irritation, or allergic response) commensurate with a reasonable benefit/risk ratio when used in the presently described manner. The phrase “therapeutically effective amount” as used herein refers to an amount of the presently described active agent effective to yield a desired therapeutic response. The actual amount administered, and the rate and time-course of administration, will depend on the nature and severity of the condition being treated. Prescription of treatment, e.g. decisions on dosage, timing, etc., is within the responsibility of general practitioners or specialists, and typically takes account of the disorder to be treated, the condition of the individual patient, the site of delivery, the method of administration and other factors known to practitioners. Examples of techniques and protocols can be found in Remington's Pharmaceutical Sciences.

As used herein, the terms “subject” or “individual” or “animal” or “patient” or “mammal,” refers to any subject, particularly a mammalian subject, for whom diagnosis, prognosis, or therapy is desired, for example, a human.

As used herein, the terms “treatment” or “treating” of a disease, disorder, or condition encompasses alleviation of at least one symptom thereof, a reduction in the severity thereof, or the delay, prevention, or inhibition of the progression thereof Treatment need not mean that the disease, disorder, or condition is totally cured. To be an effective treatment, a useful composition herein needs only to reduce the severity of a disease, disorder, or condition, reduce the severity of symptoms associated therewith, provide improvement to a patient or subject's quality of life, or delay, prevent, or inhibit the onset of a disease, disorder, or condition.

Any concentration ranges, percentage range, or ratio range recited herein are to be understood to include concentrations, percentages or ratios of any integer within that range and fractions thereof, such as one tenth and one hundredth of an integer, unless otherwise indicated.

Any number range recited herein relating to any physical feature, such as polymer subunits, size or thickness, are to be understood to include any integer within the recited range, unless otherwise indicated.

It should be understood that the terms “a” and “an” as used above and elsewhere herein refer to “one or more” of the enumerated components. It will be clear to one of ordinary skill in the art that the use of the singular includes the plural unless specifically stated otherwise. Therefore, the terms “a,” “an” and “at least one” are used interchangeably in this application.

Throughout the application, descriptions of various embodiments use “comprising” language; however, it will be understood by one of skill in the art, that in some specific instances, an embodiment can alternatively be described using the language “consisting essentially of” or “consisting of.”

For purposes of better understanding the present teachings and in no way limiting the scope of the teachings, unless otherwise indicated, all numbers expressing quantities, percentages or proportions, and other numerical values used in the specification and claims, are to be understood as being modified in all instances by the term “about.” Accordingly, unless indicated to the contrary, the numerical parameters set forth in the following specification and attached claims are approximations that may vary depending upon the desired properties sought to be obtained. At the very least, each numerical parameter should at least be construed in light of the number of reported significant digits and by applying ordinary rounding techniques.

Other terms as used herein are meant to be defined by their well-known meanings in the art.

In an embodiment, the presently described subject matter is directed to a method for treating a cell migration disease, disorder or condition in a subject, comprising administering to the subject a therapeutically effective amount of a compound of general formula I or a pharmaceutical composition comprising a compound of general formula I

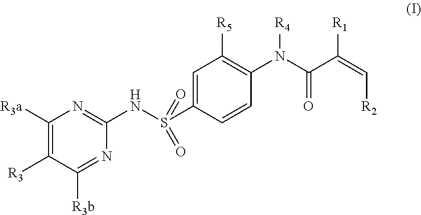

wherein

R₁ is alkyl, aryl, heteroaryl, —COR₆, —COOR₆, —NR₇R₈, —CONR₇R₈ or —NR₉COR₁₀;

R₂ is aryl or heteroaryl;

R₃, R₃a and R₃b represent H or one to three radicals selected from lower alkyl, lower alkoxy, halogen, —NR₇R₈, —COOR₆ or —CONR₇R₈ with proviso that R₃a and R₃b cannot both be H.

R₄ is H, alkyl, aryl, carbocyclyl, acyl, —O or heterocyclyl;

R₅ is H, halogen, alkyl, aryl, heteroaryl, —OR₆, —SR₆, —COR₆, —COOR₆, —NR₇R₈, —CONR₇R₈ or —NR₉COR₁₀; or R₄ and R₅ together with the carbon and nitrogen atoms to which they are attached form a 5-6 membered heterocyclic ring optionally containing a further double bond;

R₆ is H, hydrocarbyl or heterocyclyl;

R₇ and R₈ are each independently H, hydrocarbyl or heterocyclyl, or R₇ and R₈ together with the nitrogen atom to which they are attached form a 5-6 saturated heterocyclic ring, optionally containing 1 or 2 further heteroatoms selected from N, S and/or O, and wherein the further N atom is optionally substituted by lower alkyl, aralkyl, haloalkyl or hydroxyalkyl;

R₉ is H, lower alkyl or phenyl; and

R₁₀ is aryl or heteroaryl;

wherein the hydrocarbyl, heterocyclyl, aryl and heteroaryl are each independently optionally substituted by one or more radicals selected from lower alkyl, halogen, aryl, heterocyclyl, heteroaryl, nitro, epoxy, epithio, —OR₆, —SR₆, —COR₆, —COOR₆, —NR₇R₈, —CONR₇R₈, —NR₇—COR₆, —SO₃R₆, —SO₂R₆, —SO₂NR₇R₈ and —NR₇SO₂R₆, wherein R₆, R₇ and R₈ are as defined above;

or an enantiomer or a pharmaceutically acceptable salt thereof.

In a further embodiment, provided is a pharmaceutical composition comprising the at least one presently described compound, and one or more members selected from the group consisting of an anti-cancer agent; an angiogenesis inhibitor; a anti-inflammatory agent; and a chemotherapeutic agent.

In another embodiment, the presently described subject matter is directed to a method of treating a cell migration disease, disorder or condition, wherein the further N atom is optionally substituted by a member selected from the group consisting of pyrrolidino, piperidino, morpholino, thiomorpholino, piperazine, and N-methylpiperazino.

In yet a further embodiment, the presently described subject matter is directed to a method of treating a cell migration disease, disorder or condition, wherein R₁ is NR₉COR₁₀; R₂ is an optionally substituted heteroaryl; R₃, R₃a and R₃b are H or one to three alkyl radicals; with proviso that R3a and R3b cannot both be H; R₄ is H, alkyl, carbocyclyl, aryl, acyl, —O or heterocyclyl; R₅ is H, halogen, alkyl, aryl, heteroaryl, —OR₆, —SR₆, —COR₆, —COOR₆, —NR₇R₈, —CONR₇R₈ or —NR₉COR₁₀; or R₄, the nitrogen atom to which it is attached and R₅ form a 5-6 membered heterocyclic ring; R₆ is H, alkyl, aryl or heterocyclyl; R₇ and R₈ each independently is H, alkyl, aryl or heterocyclyl, or R₇ and R₈ together with the nitrogen atom to which they are attached form a saturated 5-6 heterocyclic ring, optionally containing 1 or 2 further heteroatoms selected from N, S and/or O, and wherein said further N atom is optionally substituted by lower alkyl, optionally substituted by phenyl, halogen or hydroxy; R₉ is H, lower alkyl or phenyl; and R₁₀ is aryl or heteroaryl; wherein the alkyl, carbocyclyl, heterocyclyl, aryl and heteroaryl are each independently optionally substituted by one or more radicals selected from halogen, hydrocarbyl, heterocyclyl, nitro, epoxy, epithio, OR, —SR, —COR, —COOR, —NRR′, —CONRR′, —NRCOR′, —SO₃R, —SO₂R, —SO₂NRR′ and —NRSO₂R, wherein R and R′, are each independently H, hydrocarbyl or heterocyclyl, or R and R′ together with the nitrogen atom to which they are attached form a saturated heterocyclic ring, optionally containing 1 or 2 further heteroatoms selected from N, S and/or O, and wherein the further N atom is optionally substituted by lower alkyl, aralkyl, haloalkyl or hydroxyalkyl.

In an embodiment, the presently described subject matter is directed to a method of treating a cell migration disease, disorder or condition, wherein the pharmaceutical composition optionally comprises a pharmaceutically acceptable carrier.

In another embodiment, the presently described subject matter is directed to a method of treating a cell migration disease, disorder or condition, wherein the further N atom is optionally substituted with a member selected from the group consisting of pyrrolidino, piperidino, morpholino, thiomorpholino, piperazine, and N-methylpiperazino.

In an embodiment, the presently described subject matter is directed to a method of treating a cell migration disease, disorder or condition, wherein

(i) the hydrocarbyl is a straight or branched, acyclic or cyclic, saturated, unsaturated or aromatic, hydrocarbyl radical, of 1-20 carbon atoms selected from an alkyl, alkenyl, alkynyl, carbocyclyl, aryl or an aralkyl radical;

the alkyl is a straight or branched alkyl of 1 to 10 carbon atoms (C₁-C₁₀ alkyl), optionally interrupted by one or more heteroatoms selected from O, S and/or N, and/or substituted by one or more radicals selected from the group consisting of halogen, aryl, heteroaryl, heterocyclyl, nitro, epoxy, epithio, —OR, —SR, —COR, —COOR, —NRR′, —CONRR′, —NRCOR′, —SO₃R, —SO₂R, —SO₂NRR′ and —NRSO₂R, wherein R and R′, are each independently H, hydrocarbyl or heterocyclyl, or R and R′ together with the nitrogen atom to which they are attached form a saturated 5-7 membered heterocyclic ring, optionally containing 1 or 2 further heteroatoms selected from N, S and/or O, the further N atom is optionally substituted by hydrocarbyl;

the carbocyclyl is a saturated C₅-C₆ cycloalkyl or partially unsaturated C₅-C₆ cycloalkenyl radical selected from cyclopropyl, cyclobutyl, cyclopentyl, cyclopentenyl, cyclohexyl and cyclohexenyl, optionally substituted by one or more radicals selected from the group consisting of halogen, hydrocarbyl, heterocyclyl, nitro, epoxy, epithio, OR, —SR, —COR, —COOR, —NRR′, —CONRR′, —NRCOR′, —SO₃R, —SO₂R, —SO₂NRR′ and —NRSO₂R, wherein R and R′, are each independently H, hydrocarbyl or heterocyclyl, or R and R′ together with the nitrogen atom to which they are attached form a saturated heterocyclic ring, optionally containing 1 or 2 further heteroatoms selected from N, S and/or O, and wherein the further N atom is optionally substituted by hydrocarbyl;

the aryl is a substituted or unsubstituted monocyclic, bicyclic or tricyclic aromatic carbocyclic radical of 6 to 14 carbon atoms, selected from phenyl, biphenyl, naphtyl, or anthracenyl;

(ii) the heterocyclyl is a saturated or partially unsaturated, optionally substituted, monocyclic, bicyclic or tricyclic heterocycle, of 3 to 12 ring members, of which one to three atoms is a heteroatom selected from O, S and/or N; and

(iii) the heteroaryl is a substituted or unsubstituted mono- or poly-cyclic heteroaromatic ring containing one to three heteroatoms selected from O, S and/or N.

In yet another embodiment, the presently described subject matter is directed to a method of treating a cell migration disease, disorder or condition, wherein

the hydrocarbyl is a straight or branched, acyclic or cyclic, saturated, unsaturated or aromatic, hydrocarbyl radical, of 1 to 10 carbon atoms; and/or

the alkyl is a C₁-C₄ alkyl selected from methyl, ethyl, n-propyl, isopropyl, sec-butyl or tert-butyl; and/or

the aryl is a substituted or unsubstituted monocyclic, bicyclic or tricyclic aromatic carbocyclic radical of 6 to 10 carbon atoms; and/or

the heterocyclyl is a saturated or partially unsaturated, optionally substituted, monocyclic, bicyclic or tricyclic heterocycle, of 5 to 10 ring members, of which one to three atoms is a heteroatom selected from O, S and/or N; and/or

the heteroaryl is selected from the group consisting of pyrrolyl, furyl, thienyl, pyrazolyl, imidazolyl, oxazolyl, thiazolyl, pyridyl, quinolinyl, isoquinolinyl, pyridazinyl, pyrimidinyl, pyrazinyl, 1,3,4-triazinyl, 1,2,3-triazinyl, 1,3,5-triazinyl, benzofuryl, isobenzofuryl, indolyl, imidazo[1,2-a]pyridyl, benzimidazolyl, benzthiazolyl, benzoxazolyl, and benzodiazepinyl.

In yet another embodiment, the presently described subject matter is directed to a method of treating a cell migration disease, disorder or condition, wherein

the hydrocarbyl is a straight or branched, acyclic or cyclic, saturated, unsaturated or aromatic, hydrocarbyl radical, of 1 to 6 carbon atoms; and/or

the alkyl is methyl; and/or

the heterocyclyl is a saturated or partially unsaturated, optionally substituted, monocyclic, bicyclic or tricyclic heterocycle, of 5 to 6 ring members, of which one to three atoms is a heteroatom selected from O, S and/or N; and/or

the heteroaryl is thienyl.

In a further embodiment, the presently described subject matter is directed to a method of treating a cell migration disease, disorder or condition, wherein

the hydrocarbyl is a straight or branched, acyclic or cyclic, saturated, unsaturated or aromatic, hydrocarbyl radical, of 2 to 3 carbon atoms; and/or

the heterocyclyl is a member selected from the group consisting of dihydrofuryl, tetrahydrofuryl, dihydrothienyl, pyrrolydinyl, pyrrolynyl, dihydropyridyl, piperidinyl, piperazinyl, morpholino, and 1,3-dioxanyl.

In an embodiment, the presently described subject matter is directed to a method of treating a cell migration disease, disorder or condition, wherein the compound of general formula I is a compound of formula Ia or Ib:

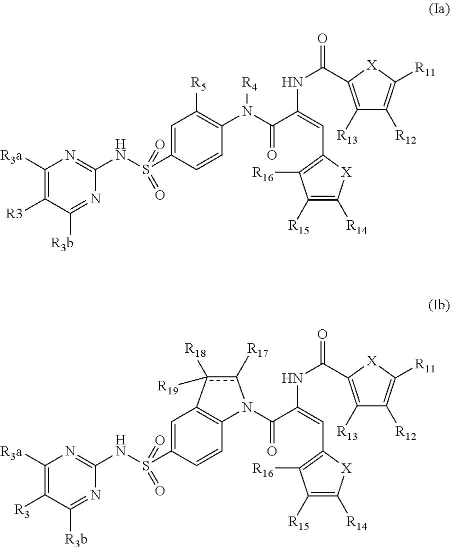

wherein

X is O, S or NH;

R₃, R₃a and R₃b are H or one to three (C₁-C₄) alkyls; with proviso that R3a and R3b cannot both be H;

R₄ is H or (C₁-C₄) alkyl;

R₅ is H or optionally substituted (C₁-C₆) alkyl;

and R₁₁ to R₁₉, are each independently selected from H, lower alkyl, halogen, aryl, heterocyclyl, heteroaryl, nitro, epoxy, epithio, —OR₆, —SR₆, —COR₆, —COOR₆, —NR₇R₈, —CONR₇R₈, —NR₇—COR₆, —SO₃R₆, —SO₂R₆, —SO₂NR₇R₈ and —NR₇SO₂R₆, wherein R₆, R₇ and R₈ are each independently H, alkyl, aryl or heterocyclyl, or R₇ and R₈ together with the nitrogen atom to which they are attached form a saturated heterocyclic ring, optionally containing 1 or 2 further heteroatoms selected from N, S and/or O, and wherein the further N atom is optionally substituted by lower alkyl, optionally substituted by phenyl, halogen or hydroxy; and

the dotted line in formula Ib represents an optional double bond.

In a further embodiment, the presently described subject matter is directed to a method of treating a cell migration disease, disorder or condition, wherein in the compound of formula Ia, X is S, R₃, R₃a and R₃b are H or one to three methyl groups; with proviso that R₃a and R₃b cannot both be H, R₄ is H, R₅ is H or methyl and R₁₁ to R₁₆ are H.

In yet a further embodiment, the presently described subject matter is directed to a method of treating a cell migration disease, disorder or condition, wherein the compound of formula Ia is selected from the compounds herein identified as:

compound 1 of the formula:

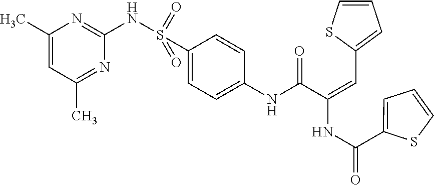

compound 2 of the formula:

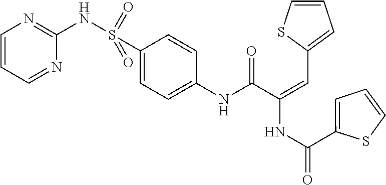

compound 3 of the formula:

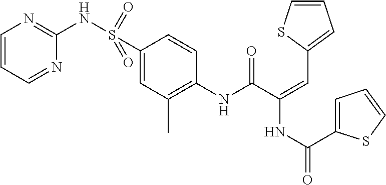

; and

compound 4 of the formula:

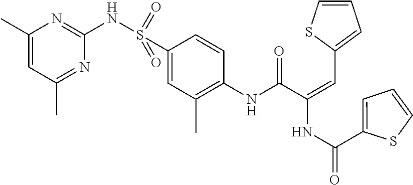

In another embodiment, the presently described subject matter is directed to a method of treating a cell migration disease, disorder or condition, wherein in the compound of formula Ib, X is S, R₃, R₃a and R₃b are H or one to three methyl groups; with proviso that R₃a and R₃b cannot both be H and R₁₁ to R₁₉ are H.

In yet another embodiment, the presently described subject matter is directed to a method of treating a cell migration disease, disorder or condition, wherein the compound of formula Ib is selected from the compounds herein identified as

compound 5 of the formula:

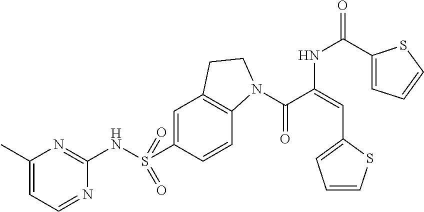

compound 6 of the formula:

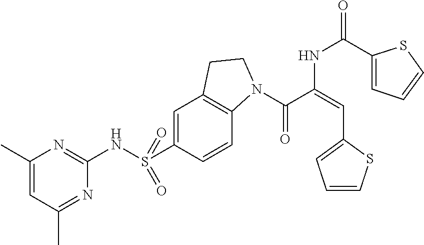

; and

compound 7 of the formula:

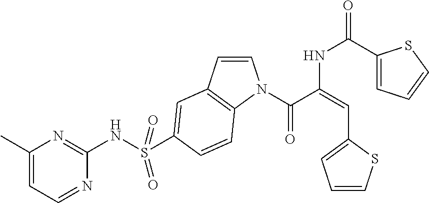

In an embodiment, the presently described subject matter is directed to a method of treating a cell migration disease, disorder or condition, wherein the disease, disorder or condition is cancer.

In another embodiment, the presently described subject matter is directed to a method of treating a cancer selected from the group consisting of anal cancer; astrocytoma; leukemia; lymphoma; head and neck cancer; liver cancer; testicular cancer; cervical cancer; sarcoma, hemangioma; esophageal cancer; eye cancer; laryngeal cancer; mouth cancer; mesothelioma; myeloma; oral cancer; rectal cancer; throat cancer; bladder cancer; breast cancer; uterine cancer; ovarian cancer; prostate cancer; lung cancer; colon cancer; pancreatic cancer; renal cell carcinoma; gastric cancer; skin cancer; basal cell carcinoma; melanoma; squamous cell carcinoma; oral squamous cell carcinoma; colorectal cancer; glioblastoma multiforme; endometrial cancer; and malignant glioma.

In yet another embodiment, the presently described subject matter is directed to a method of treating cancer, wherein the pharmaceutical composition further comprises an effective amount of at least one anti-cancer agent.

In a further embodiment, the presently described subject matter is directed to a method of treating cancer, wherein the at least one anti-cancer agent is selected from the group consisting of imatinib, dasatinib, axitinib, bosutinib, cediranib, erlotinib, gefitinib, lapatinib, lestaurtinib, nilotinib, semaxanib, sunitinib, toceranib, vandetanib, and vatalanib.

In yet a further embodiment, the presently described subject matter is directed to a method of treating cancer, further comprising administering to the subject an effective amount of at least one anti-cancer agent.

In another embodiment, the presently described subject matter is directed to a method of treating cancer, wherein the at least one anti-cancer agent is administered simultaneously with, before or after administration of the pharmaceutical composition.

In an embodiment, the presently described subject matter is directed to a method of treating cancer, wherein the pharmaceutical composition further comprises an effective amount of at least one cancer chemotherapeutic agent.

In yet another embodiment, the presently described subject matter is directed to a method of treating cancer, wherein the at least one cancer chemotherapeutic agent is selected from the group consisting of mechlorethamine; cyclophosphamide; ifosfamide; melphalan; chlorambucil; dicarbazine; streptazocine; carmustine; lomustine; semustine; chlorozotocin; busulfan; triethylenemelamine; thiotepa; hexamethylmelamine; an antimetabolite; methotrexate; fluorouracil; 5-fluorouracil; floxuridine (5′-fluoro-2′-deoxyuridine); idoxuridine; cytarabine; N-phosphonoacetyl-L-aspartate; 5-azacytidine; azaribine; 6-azauridine; pyrazofuran; 3-deazauridine; acivicin; a purine analog; thioguanine; mercaptopurine; azathioprine; pentostatin; erythrohydroxynonyladenine; a vinca alkaloid; vincristine; vinblastine; an epipodophyllotoxin; etoposide; teniposide; an antibiotic; dactinomycin; daunorubicin; doxorubicin; bleomycin sulfate; plicamycin; mitomycin; an enzyme; L-asparaginase; a platinum coordination complex; cisplatin; carboplatin; hydroxyurea; procarbazine; mitotane; a hormone; an adrenocorticosteroid; prednisone; prednisolone; aminoglutethimide; a progestin; hydroxyprogesterone caproate; medroxyprogesterone acetate; megesterol acetate; estrogen; an androgen; diethylstilbestrol; fluoxymesterone; ethynyl estradiol; an antiestrogen; tamoxifen; a gonadotropin-releasing hormone analog; and leuprolide.

In a further embodiment, the presently described subject matter is directed to a method of treating cancer, further comprising administering to the subject an effective amount of at least one cancer chemotherapeutic agent.

In an embodiment, the presently described subject matter is directed to a method of treating cancer, wherein the at least one cancer chemotherapeutic agent is administered simultaneously with, before or after administration of the pharmaceutical composition.

In another embodiment, the presently described subject matter is directed to a method of treating cancer, further comprising administering to the subject radiation therapy.

In an embodiment, the presently described subject matter is directed to a method of treating cancer, wherein radiation therapy is administered simultaneous with, before or after administration of the pharmaceutical composition.

In another embodiment, the presently described subject matter is directed to a method of treating a cell migration disease, disorder or condition, wherein the cell migration disease, disorder or condition is an inflammatory condition.

In a further embodiment, the presently described subject matter is directed to a method of treating an inflammatory condition selected from the group consisting of pulmonary fibrosis; ischaemic heart disease; Crohn's disease; dermatomyositis; diabetes mellitus; Guillain-Barre syndrome; hashimoto's disease; idiopathic thrombocytopenic purpura; mixed connective tissue disease; myasthenia gravis; narcolepsy; pemphigus vulgaris; pernicious anaemia; polymyositis; primary biliary cirrhosis; Sjogren's syndrome; temporal arteritis; ulcerative colitis; vasculitis; Wegener's granulomatosis; systemic lupus erythematosus; lupus nephritis; Goodpasture's syndrome; haemolytic anaemia; thyrotoxicosis; scleroderma; asthma; rheumatoid arthritis; osteoarthritis; septicaemia; artherosclerosis; chronic renal disease; inflammatory bowel disease; vasculitis; peritonitis; giant papillary conjunctivitis; uveitis; seasonal allergic conjunctivitis; chronic prostatitis; glomerulonephritis; hypersensitivities; inflammatory bowel diseases; pelvic inflammatory disease; reperfusion injury; transplant rejection; Chediak-Higashi syndrome; chronic granulomatous disease; urinary tract inflammatory conditions; interstitial cystitis; ulcerative colitis; systemic sclerosis; dermatomyositis; polymyositis; and inclusion body myositis.

In yet another embodiment, the presently described subject matter is directed to a method of treating an inflammatory condition, wherein the pharmaceutical composition further comprises at least one anti-inflammatory agent.

In another embodiment, the presently described subject matter is directed to a method of treating an inflammatory condition, wherein the anti-inflammatory agent is selected from the group consisting of a corticosteroid; cortisol; aldosterone; hydrocortisone; hydrocortisone acetate; cortisone acetate; tixocortol pivalate; prednisolone; methylprednisolone; prednisone; triamcinolone acetonide; triamcinolone alcohol; mometasone; amcinonide; budesonide; desonide; flucinonide; fluocinolone acetonide; halcinonide; betamethasone; betamethasone sodium phosphate; dexamethasone; dexamethasone dodium phosphate; flucortolone; hydrocortisone-17-butyrate; hydrocortisone-17-valerate; aclometasone dipropionate; betamethasone valerate; betamethasone dipropionate; prednicarbate; clobetasone-17-butyrate; clobetasol-17-propionate; flucortolone caproate; fluocortolone pivalate; fluprednidene acetate; a non-steroidal anti-inflammatory; a cox-2 inhibitor; nimesulide; diclofenac; licofelone; aspirin; ibuprofen; naproxen; an immune selective anti-inflammatory derivative; phenylalanine-glutamine-glycine; an herb; Harpagophytum; hyssop; ginger; turmeric; Arnica Montana; willow bark; and cannabis.

In a further embodiment, the presently described subject matter is directed to a method of treating an inflammatory condition, further comprising administering to the subject an effective amount of at least one anti-inflammatory agent.

In yet a further embodiment, the presently described subject matter is directed to a method of treating an inflammatory condition, wherein the at least one anti-inflammatory agent is administered simultaneous with, before or after administration of the pharmaceutical composition.

In another embodiment, the presently described subject matter is directed to a method of treating a cell migration disease, disorder or condition, wherein the cell migration disease, disorder, or condition is an excessive angiogenesis condition.

In an embodiment, the presently described subject matter is directed to a method of treating an excessive angiogenesis condition selected from the group consisting of diabetic blindness; chronic inflammation; arthritis; age-related macular degeneration; retinopathy; rheumatoid arthritis; osteoarthritis; Crohn's disease; psoriasis; cancer; Alzheimer's disease; restenosis; pulmonary fibrosis; asthma; angiofibroma; neovascular glaucoma; arteriovenous malformations; nonunion fractures; lupus and a connective tissue disorder; Osler-Weber syndrome; atherosclerotic plaques; corneal graft neovascularization; pyogenic granuloma; retrolental fibroplasias; scleroderma; granulations; hemangioma; trachoma; hemophilic joints; peritoneal endometriosis; adiposity; and vascular adhesions.

In another embodiment, the presently described subject matter is directed to a method of treating an excessive angiogenesis condition, wherein the pharmaceutical composition further comprises at least one angiogenesis inhibitor.

In an embodiment, the presently described subject matter is directed to a method of treating an excessive angiogenesis condition, wherein the at least one angiogenesis inhibitor is selected from the group consisting of bevacizumab, sunitinib, sorafenib, thalidomide, lenalidomide, panitumumab, cetuximab, and erlotinib.

In a further embodiment, the presently described subject matter is directed to a method of treating a cell migration disease, disorder or condition, further comprising administering to the subject an effective amount of at least one member selected from the group consisting of an anti-cancer agent, an angiogenesis inhibitor and an anti-inflammatory agent.

In an embodiment, the presently described subject matter is directed to a method of treating a cell migration disease, disorder or condition, further comprising administering to the subject an effective amount of an anti-cancer agent and an angiogenesis inhibitor, and optionally an anti-inflammatory agent.

In a further embodiment, the presently described subject matter is directed to a method of treating cancer, further comprising administering to the subject an effective amount of an anti-cancer agent and an angiogenesis inhibitor, and optionally an anti-inflammatory agent. The anti-cancer agent and angiogenesis inhibitor, and optionally the anti-inflammatory agent, can be administered along with the presently described compounds as a unitary pharmaceutical composition, or can be administered separately from the presently described compounds. Such separate administration can, for each agent or inhibitor, be before, simultaneous with or after administration of the presently described compounds.

In an embodiment, the presently described subject matter is directed to a method of treating a cell migration disease, disorder or condition, wherein the pharmaceutical composition further comprises an effective amount of at least one member selected from the group consisting of an anti-cancer agent, an angiogenesis inhibitor, and an anti-inflammatory agent.

In another embodiment, the presently described subject matter relates to the use of the compounds of the general formula I above for the treatment of breast, colon, prostate and pancreatic cancer.

The presently described compounds can be readily produced by methods well known to those of ordinary skill in the art, without undue experimentation.

The pharmaceutical compositions for use in accordance with the presently described subject matter may be formulated in conventional manner using one or more physiologically acceptable carriers or excipients. Thus, the compounds and their physiologically acceptable salts and solvates may be formulated by conventional methods as described, for example, in Remington's Pharmaceutical Sciences, Meade Publishing Co., Easton, Pa., for administration by a variety of routes of administration, including systemic and topical or localized administration.

For systemic administration, injection is preferred, including intramuscular, intravenous, intraperitoneal, and subcutaneous. For injection, the compounds of the presently described subject matter can be formulated in liquid solutions, for example in physiologically compatible buffers such as Hank's solution or Ringer's solution. In addition, the compounds may be formulated in solid form and redissolved or suspended immediately prior to use. Lyophilized forms are also included.

For oral administration, the pharmaceutical compositions may take the form of, for example, tablets or capsules prepared by conventional means with pharmaceutically acceptable excipients such as binding agents; fillers (e.g., lactose, microcrystalline cellulose or calcium hydrogen phosphate); lubricants (e.g., magnesium stearate, talc or silica); disintegrants (e.g., potato starch or sodium starch glycolate); or wetting agents (e.g., sodium lauryl sulphate). The tablets may be coated by methods well known in the art. Liquid preparations for oral administration may take the form of, for example, solutions, syrups or suspensions, or they may be presented as a dry product for constitution with water or other suitable vehicle before use. Such liquid preparations may be prepared by conventional means with pharmaceutically acceptable additives such as suspending agents (e.g., sorbitol syrup, cellulose derivatives or hydrogenated edible fats); emulsifying agents (e.g., lecithin or acacia); non-aqueous vehicles (e.g., ationd oil, oily esters, ethyl alcohol or fractionated vegetable oils); and preservatives (e.g., methyl or propyl-p-hydroxybenzoates or sorbic acid). The preparations may also contain buffer salts, flavoring, coloring and sweetening agents as appropriate.

Preparations for oral administration may be suitably formulated to give controlled release of the active compound. For buccal administration the compositions may take the form of tablets or lozenges formulated in conventional manner. For administration by inhalation, the compounds for use according to the presently described subject matter are conveniently delivered in the form of an aerosol spray presentation from pressurized packs or a nebuliser, with the use of a suitable propellant, e.g., dichlorodifluoromethane, trichlorofluoromethane, dichlorotetra-fluoroethane, carbon dioxide or other suitable gas. In the case of a pressurized aerosol the dosage unit may be determined by providing a valve to deliver a metered amount. Capsules and cartridges of e.g., gelatin for use in an inhaler or insufflator may be formulated containing a powder mix of the compound and a suitable powder base such as lactose or starch.

The compounds may be formulated for parenteral administration by injection, e.g., by bolus injection or continuous infusion. Formulations for injection may be presented in unit dosage form, e.g., in ampoules or in multi-dose containers, with an added preservative. The compositions may take such forms as suspensions, solutions or emulsions in oily or aqueous vehicles, and may contain formulatory agents such as suspending, stabilizing and/or dispersing agents. Alternatively, the active ingredient may be in powder form for constitution with a suitable vehicle, e.g., sterile pyrogen-free water, before use.

The compounds may also be formulated in rectal compositions such as suppositories or retention enemas, e.g., containing conventional suppository bases such as cocoa butter or other glycerides.

In addition to the formulations described previously, the compounds may also be formulated as a depot preparation. Such long acting formulations may be administered by implantation (for example subcutaneously or intramuscularly) or by intramuscular injection. Thus, for example, the compounds may be formulated with suitable polymeric or hydrophobic materials (for example as an emulsion in an acceptable oil) or ion exchange resins, or as sparingly soluble derivatives, for example, as a sparingly soluble salt.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration bile salts and fusidic acid derivatives. In addition, detergents may be used to facilitate permeation. Transmucosal administration may be through nasal sprays or using suppositories. For topical administration, the presently described compounds are formulated into ointments, salves, gels, or creams as generally known in the art. A wash solution can be used locally to treat an injury or inflammation to accelerate healing.

The compositions may, if desired, be presented in a pack or dispenser device which may contain one or more unit dosage forms containing the active ingredient. The pack may for example comprise metal or plastic foil, such as a blister pack. The pack or dispenser device may be accompanied by instructions for administration.

The presently described subject matter now being generally described, it will be more readily understood by reference to the following examples, which are included merely for purposes of illustration of certain aspects and embodiments of the presently described subject matter, and are not intended to limit the presently described subject matter.

### EXAMPLES

### Example 1

### Synthesis of Compound 1 (PRT7000467)

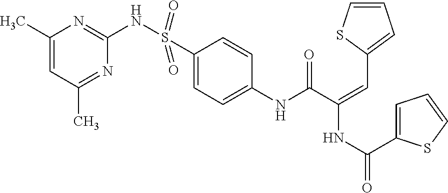

To a solution of glycine (7.0 g, 93 mmol) and potassium carbonate (13.8 g, 100 mmol) in water (100 mL) thiophene-2-carbonyl chloride (7.3 g, 50 mmol) was added over a period of 30 mins with stirring. The resulting solution was stirred for 1 hr, washed with diethyl ether (2×30 mL,) and acidified with conc. HCl. After being cooling for 1 hr in an ice-bath, the precipitate was filtered off, washed with ice-water, and dried in air to yield 7.0 g (76%) of the acid as presented below.

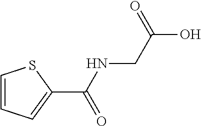

The suspension of acid from the previous step (7.0 g, 38 mmol), sodium acetate (3.1 g, 38 mmol) and acetic anhydride (11.6 g, 114 mmol) was heated on a steam-bath for 1 hr with stirring. The mixture became orange and solidified during the reaction. The cooled solid was stirred with water (50 mL) for 15 min and the resulting precipitate was filtered off, washed with ice-water and ice-cooled ethanol, and dried in air to yield 6.2 g (63%) of the azalactone as presented below as an orange solid.

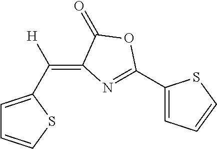

The suspension of azalactone from the previous step (2.8 g, 11 mmol) and 4-amino-N-(4,6-dimethylpyrimidin-2-yl)benzenesulfonamide (2.8 g, 10 mmol) in glacial acetic acid (40 mL) was stirred under reflux for 1 hr. The solids were dissolved and then resulted in a yellow precipitate. After cooling, the latter was filtered off, washed successively with glacial acetic acid, ethanol, and then with diethyl ether and dried in air to yield 3.7 g (69%) of the amide, Compound 1, as a light yellow powder.

### Example 2

### Synthesis of Compound 3 (PRT7081582)

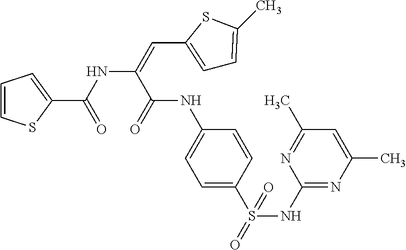

To a solution of glycine (7.0 g, 93 mmol) and potassium carbonate (13.8 g, 100 mmol) in water (100 mL) thiophene-2-carbonyl chloride (7.3 g, 50 mmol) was added over a period of 30 mins with stirring. The resulting solution was stirred for 1 hr, washed with diethyl ether (2×30 mL,) and acidified with conc. HCl. After cooling for 1 hr in an ice-bath, the precipitate was filtered off, washed with ice-water, and dried in air to yield 7.0 g (76%) of the acid as presented below.

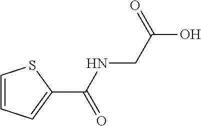

The suspension of acid from previous step (7.0 g, 38 mmol), sodium acetate (3.1 g, 38 mmol) and acetic anhydride (11.6 g, 114 mmol) was heated on a steam-bath for 1 hr with stirring. The mixture became orange and solidified during the reaction. The cooled solid was stirred with water (50 mL) for 15 min and the resulting precipitate was filtered off, washed with ice-water and ice-cooled ethanol, and dried in air to yield 6.2 g (63%) of the azalactone presented below as an orange solid.

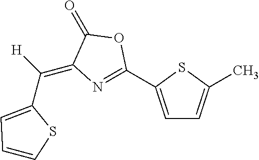

The suspension of azalactone from the previous step (2.8 g, 11 mmol) and 4-amino-N-(4,6-dimethylpyrimidin-2-yl)benzenesulfonamide (2.8 g, 10 mmol) in glacial acetic acid (40 mL) was stirred under reflux for 1 hr. The solids were dissolved and then resulted in a yellow precipitate. After cooling, the latter was filtered off, washed successively with glacial acetic acid, ethanol, and then with diethyl ether and dried in air to yield 3.7 g (69%) of the amide, Compound 3, as a light yellow powder.

### Example 3

### POSH Inhibitors Inhibit Cell Migration and Soft Agar Colony Formation

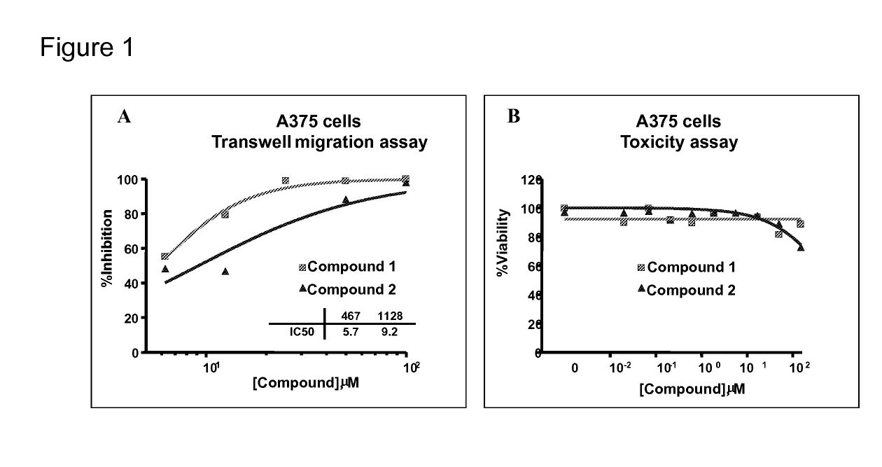

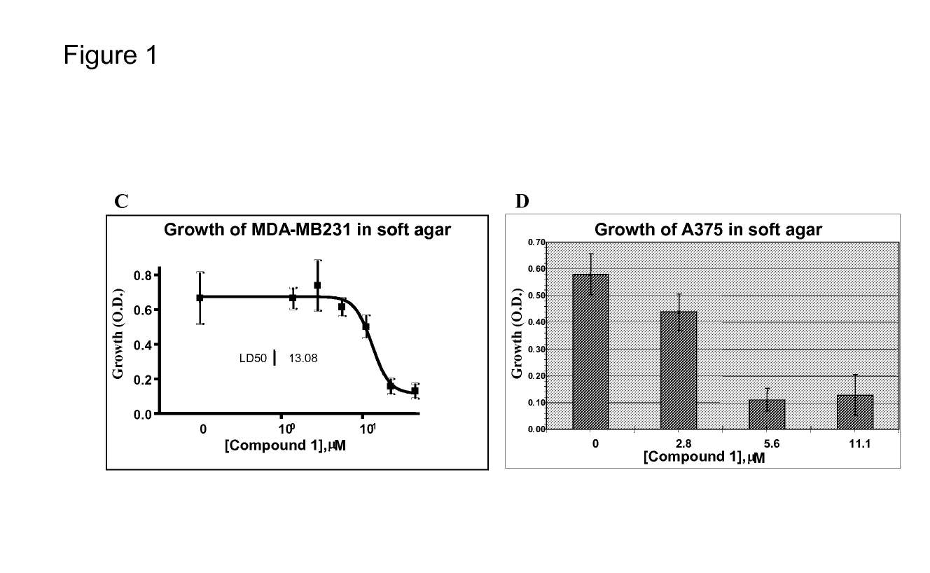

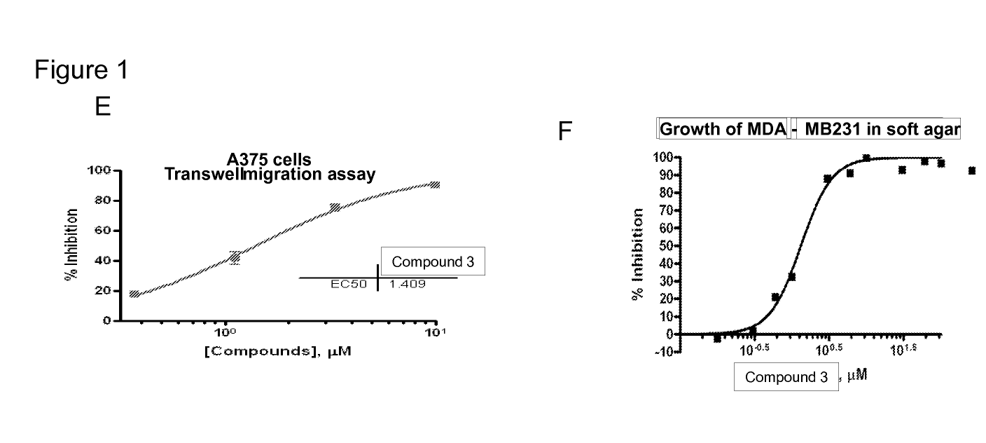

A375 melanoma cells (CRL-1619, American Type Culture Collection (“ATCC”), Manassas Va., USA)) grown in RPMI medium supplement with 10% FBS were starved for 24 hours in FBS free medium. After starvation of 24 hr, 5×10⁴ cells in FBS free medium were placed in the upper chamber of 24-well, Transwell apparatus (CORNING TRANS WELL® polycarbonate membrane inserts, 5 μm pore size) with solvent (DMSO/PEG400) or different concentrations of Compound 1 (PRT7000467), Compound 2 (PRT7041128), or Compound 3 (PRT7081582). Medium containing FBS and compounds was added to the bottom chamber. Twenty-four hours later the melanoma cells from the upper chamber were removed with a cotton swab. Cells that migrated to the lower side of the membrane were stained with Calcein-AM (Sigma) and images were taken with fluorescence microscope. The number of migrating cells was quantified by ImageJ software and the IC50 values were calculated using Prism software (Graphpad) (FIG. 1A,1E).

Compound 1 (PRT7000467), Compound 2 (PRT7041128), and Compound 3 (PRT7081582) are not toxic to cells grown in two-dimension. A375 cells were cultured with RPMI and 10% FBS. The cells were treated with different concentrations of Compound 1 (PRT7000467) and Compound 2 (PRT7041128), then the viability of the cells was determined seventy-two hours post treatment using WST-1 reagent (Roche) (FIG. 1B)

Compound 1 (PRT7000467) inhibits soft agar colony formation of A375 melanoma cells and MDA-MB231 breast cancer cells. The assay was done using CYTOSELECT™ 96-Well In Vitro Tumor Sensitivity Assay kit from Cell Biolabs, Inc. (San-Diego, Calif.). The cells were grown for 14 days with RPMI and 10% FBS medium in the presence of the indicated concentration of Compound 1 (PRT7000467). The quantification of the colony formation on soft agar was done using MTT reagent after solubilization of the agar (all the materials are supplied in the kit). The IC50 value was calculated using Prism software. The migration of A375 melanoma cells in Transwell assay was inhibited by POSH inhibitors, Compound 1(PRT7000467), Compound 2 (PRT7041128), and Compound 3 (PRT7081582), with IC50 in the migration assay of 5.7 μM, 9.2 μM, and 1.409 μM, respectively (FIGS. 1A and 1E). This effect on cell migration was not due to toxicity since the compounds are not toxic to cells and their LD50 is higher than 150 μM (FIG. 1B). Compound 1 (PRT7000467) and Compound 3 (PRT7081582) inhibit soft agar colony formation of A375 melanoma cells (FIGS. 1C and 1F respectively). Furthermore, it can be seen that Compound 1 inhibits soft agar colony formation of MDA-MB231 breast cancer cells (FIG. 1D). The LD50 of the compound in the soft agar colony formation assay was 13.08 μM for MDA-MB231 cells and between 2.8-5.6 μM in A375 cells.

The results from this experiment indicate that Compound 1 and Compound 3 have anti-migratory activity on A375 cells at a concentration which is well below the toxic concentration of these compounds. Further, Compound 2 also has anti-migratory activity on A375 cells at a concentration which is well below the toxic concentration of this compound. These results indicate that Compounds 1, 2 and 3 can be used for indications involving cell migration, including for example, cancer, angiogenesis indications and inflammatory conditions, without causing any significant cellular toxicity.

### Example 4

### Compound 1 Inhibits the Migration of Various Cancer Cell Lines

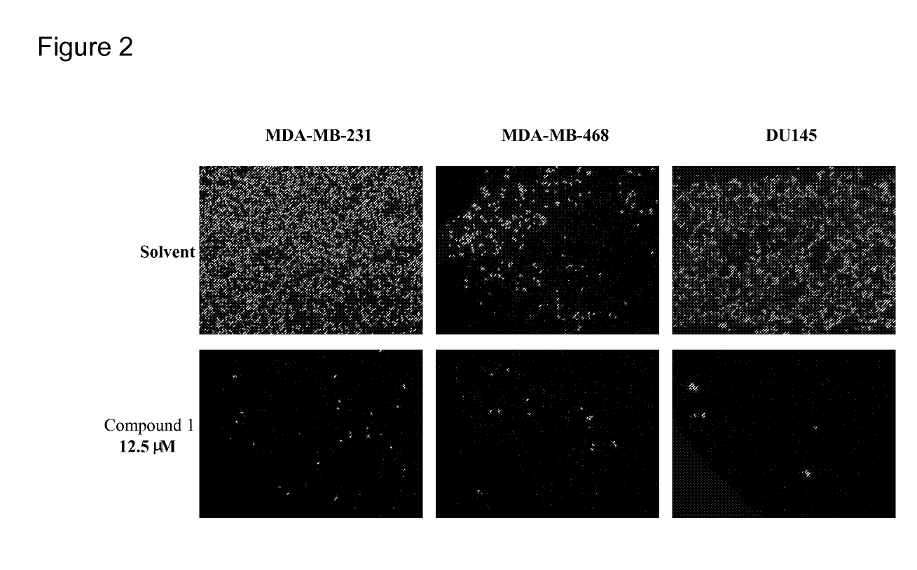

MDA-MB-231 (HTB-26, ATCC, Manassas, Va., USA) and MDA-MB-468 (HTB-132, ATCC, Manassas, Va., USA) breast cancer cell lines and DU 145 (HTB-81, ATCC, Manassas, Va., USA) prostate cancer cell line grown in RPMI medium supplement with 10% FBS were starved for 24 hours in medium without FBS. After 24 hours, 5×10⁴ cells in FBS free medium were placed in the upper chamber of 24-well Transwell apparatus (CORNING TRANSWELL® polycarbonate membrane inserts, 8 μm pore size) with solvent (DMSO/PEG400) or 12.5 μM Compound 1 as indicated. Medium with FBS and Compound 1 was added to the bottom chamber. Twenty-four hours later the cells from the upper chamber were removed with a cotton swab. Cells that migrated to the lower side of the membrane were stained with Calcein-AM (Sigma) and images were taken with a fluorescence microscope. The results show that the inhibitory effect of POSH inhibitor on cell migration is not restricted to A375 cells. Compound 1 (PRT7000467) also inhibits the migration of other cancer cells such as MDA-MB-231, MDA-MB-468 breast cancer cells and DU145 prostate cancer cell line (FIG. 2).

The inhibitory effect of Compound 1 was reexamined on various cell-lines and was measured by Transwell assay.

A375 (melanoma) (CRL-1619, ATCC, Manassas, Va., USA), A431 (melanoma) (CRL-2592, ATCC, Manassas, Va., USA), A2058 (melanoma) (CRL-11147, ATCC, Manassas, Va., USA), MDA-MB-231 (breast) (HTB-26, ATCC, Manassas, Va., USA), LS175T (colon) (ATCC, Manassas, Va., USA), HT29 (colon) (HTB-38, ATCC, Manassas, Va., USA), PC3 (prostate) (CRL-1435, ATCC, Manassas, Va., USA), Panc03.27 (pancreatic) (ATCC, Manassas, Va., USA), 4T1 (breast-mouse) (CRL-2539, ATCC, Manassas. Va., USA), D122 (lung-mouse) (the Weizmann Institute of Science), D122-luc (lung-mouse) (the Weizmann Institute of Science), and B16 F10.9 (melanoma-mouse) cells (Caliper Life Sciences, Hopkinton Mass.), were grown in RPMI medium supplemented with 10% FBS. HUVEC cells were grown in Endothelial Cell Growth Medium 2 and THP1 cells were grown in RPMI medium supplemented with 10% FBS, 2 mM L-Glutamine, 1.5 g/L sodium bicarbonate, 10 mM Hepes, 1 mM sodium pyruvate, 4.5 g/L glucose and 0.05 mM 2-mercaptoethanol. The cells were starved for 24 hours in medium without FBS. At the end of the starvation, 5×10⁴ cells in medium without FBS were placed in the upper chamber of 24-well, Transwell apparatus (CORNING TRANSWELL® polycarbonate membrane inserts, 5 μm pore size for A365 and THP1 cells and 8 μm pore size for all the other cells) with solvent (DMSO/PEG400) or different concentrations of compound 1 (PRT7000467). Medium with FBS and compounds was added to the bottom chamber. Twenty-four hours later the cells from the upper chamber were removed with a cotton swab. Cells that migrated to the lower side of the membrane were stained with Calcein-AM (Sigma) and images were taken with a fluorescence microscope. The number of migrating cells was quantified by ImageJ software and the IC50 values were calculated using Prism software (Graphpad). Results are presented in Table 1 which shows that compound 1 is active against all the cells that were tested with the exception of the THP1 monocytes where its activity was much weaker.

TABLE 1

| Cell Line | Origin | 7000467 IC50 (μM) | Repeats |
|---|---|---|---|
| A375 | Melanoma | 1.7 | 6 |
| A431 | Melanoma | 1.1 | 1 |
| A2058 | Melanoma | 3.5 | 1 |
| MDA-MB231 | Breast | 0.5 | 3 |
| LS175T | Colon | 1 | 1 |
| HT29 | Colon | 1.5 | 1 |
| PC3 | Prostate | 6.3 | 2 |
| Panc 03.27 | Panceatic | 1.0 | 1 |
| 4T1 | Breast (mouse) | 0.8 | 2 |
| D122 | Lung (mouse) | 1.4 | 4 |
| D122-luc | Lung (mouse) | 1.6 | 3 |
| B16 F10.9 | Melanoma (mouse) | 0.2 | 1 |
| HUVEC | Endothelial | 2.9 | 2 |
| THP1 | Monocyte | >30 | 3 |

### Example 5

### Compound 1 (PRT7000467) Inhibits HUVEC Cells Tube-Formation

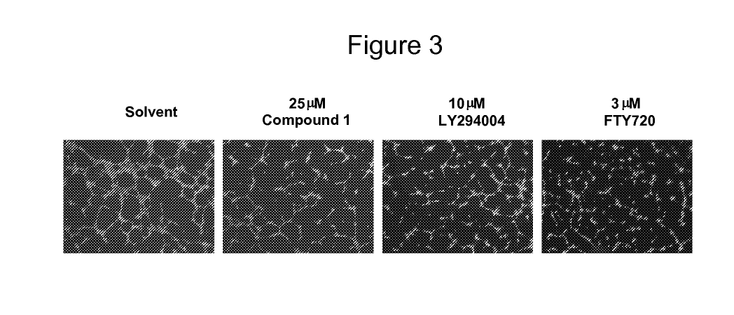

Human umbilical vein endothelial cells (HUVEC) were grown routinely in Endothelial Cell Growth Medium 2 with SupplementMix (PromoCell, Germany) containing 15% fetal bovine serum. For Tube-formation assay, 1×10⁴ cells, in the above medium containing 5% fetal bovine serum, were seeded on top of Cultrex reduced growth factor Basement Membrane Extract (BME) (R&D Systems, Minneapolis, Minn.) supplemented with 50 ng/ml VEGF (Prospec, Israel). The cells were treated with either solvent (50% DMSO/50% PEG400), 25 μM compound 1 (PRT7000467), 10 μM LY294004 (Cayman Chemical, Ann Arbor, Mich.) or 3 μM FTY720 (Cayman Chemical, Ann Arbor, Mich.) as indicated. LY294004 is a PI3K inhibitor and FTY720 is a modulator of sphingosine-1-phosphate receptors, both of them served as positive controls. Six hours later the cells were stained with Calcein-AM (Sigma, Israel) and images were taken with a fluorescence microscope. The results show that compound 1 (PRT7000467) inhibits the formation of tube-like structure by the HUVEC cells, an in-vitro assay that mimics in-vivo angiogenesis (FIG. 3).

### Example 6

### Anti-Metastasis Migration Assay

Compound 1 and Compound 3 were tested for efficacy in inhibiting lung metastases in female B6D2F 1 mice implanted with B16F10 murine melanoma cells. Tumors were initiated on Day 1 (D1) by intravenously (i.v.) injecting 1×105 B16F10 tumor cells into the tail vein of test mice. Mice were sorted into three vehicle-treated control groups (n=20 or 10/group) and nine treatment groups (n=10/group). Compound 1 and Compound 3 monotherapies were administered at 10 mg/kg (2 mg/mL at 5 mL/kg) and at 20 mg/kg (4 mg/mL at 5 mL/kg), intraperitoneally (i.p.), daily for fourteen doses starting on D3 (qd×14 (start D3)). Compound 1 and Compound 3 monotherapies also were administered at 20 mg/kg (4 mg/mL at 5 mL/kg), i.p., twice daily for twenty-nine doses starting on D3 (bid×14.5 (start D3)). A control group that received test article vehicle at 5 mL/kg, i.p., qd×14 (start D3) served as a “like-treated” comparator for these groups. A control group that received test article vehicle at 10 mL/kg, i.p., qd×14 (start D3) served as a “like-treated” comparator for these groups. A positive control group received one dose of cyclophosphamide at 300 mg/kg, i.p., on Day 3. A control group received one dose of saline, intraperitoneally (i.p.), on Day 3, and served as the control for the “like-treated” comparator and positive control groups.

The study endpoint was defined as 100 metastases per lung set in animals periodically sampled from the saline control group. The study was terminated when the “look-see” animals sampled on D17 yielded a mean count of 77.0 metastatic foci. All animals remaining on study were euthanized, their metastases were counted, and the mean±SEM foci count for each group was calculated. Efficacy was determined by an analysis of percent inhibition, the percent change in the mean foci count on D17 for treated versus comparator mice, and by statistical assessment of differences in foci counts between treated and comparator mice.

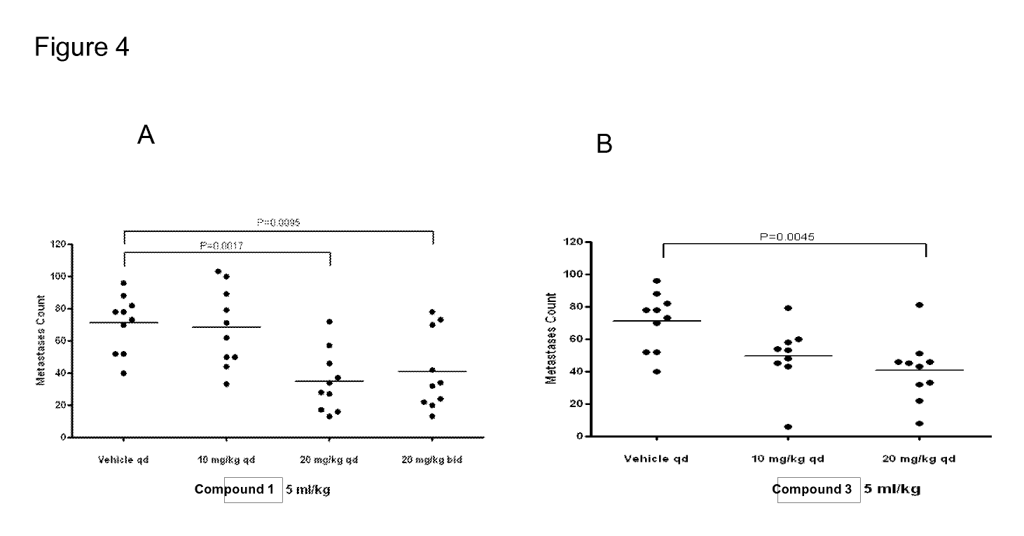

Compound 1 and Compound 3 exhibited 51% and 45% efficacy respectively, in inhibiting lung metastases in female B6D2F1 mice implanted with B 16F10 murine melanoma cells (FIGS. 4A and B).

### Example 7

Anti inflammatory assay A delayed-type hypersensitivity (DTH) reaction is an expression of cell-mediated immunity and plays a major role in the pathology and chronicity of many inflammatory disorders. Delayed-type hypersensitivity (DTH) reactions can be induced by various allergens, including oxazolone, 2,4-dinitrofluorobenzene (DNFB) and sheep red blood cells (SRBCs). One of the most characteristic DTH phenomenons is contact hypersensitivity, which is characterized by swelling and by increased tissue levels of cytokines. Contact hypersensitivity (CHS) is a T cell-mediated immune reaction in response to cutaneous sensitization and challenge with reactive haptens that are capable of binding directly to soluble and cell-associated proteins and recognized by T cells in the context of self-MHC products. The cells that recognize antigen-protein complex in the skin are the Langerhans cells (LCs). After topically allergen application, Langerhans cells (LCs), the major antigen-presenting cells (APCs) for the induction of immune responses in skin, show enhanced expression of surface MHC class II molecules, and start to emigrate from the skin to regional lymph nodes where specific lymphocyte activation is thought to occur. After a second contact with the hapten, T cells are first recruited into tissues and then activated by antigen-presenting cells to produce cytokines that mediate local inflammation. Myeloperoxidase (MPO) is an enzyme exclusively present in neutrophil granules, which is commonly used as an index of granulocyte infiltration, and its inhibition is indicative of an anti-inflammatory action.

The goal of this study is to examine the effects of Compounds 1 and 3 on oxazolone-induced DTH.

Animal:
Animal species and strain: 50 BALB/c mice
Sex and age and weight: Female, 19-21 g, 6-8 weeks
Breeder/supplier: Shanghai SLAC Laboratory Animal Co. Ltd.

Animal Quarters and Husbandry:
Test Facility: PharmaLegacy Laboratories Vivarium
Adaptation: Not less than 7 days
Room: Conventional Room
Room temperature: (19-26)° C.
Room relative humidity: 40-70%
Light cycle: Fluorescent light for 12-hour light (8:00-20:00) and 12-hour dark
Animal housing: 5 mice/cage by treatment group
Food: Free access to food (irradiated, Shanghai SLAC Laboratory Animal Co. Ltd., China)
Water: Free access to water (municipal tap water filtered by Molanimal Ultrapure Water System)

Reagents:
Oxazolone: Sigma-Aldrich. (St. Louis, Mo., USA), Cat: E0753, Lot: 124K3690.
Pentobarbital sodium: Shanghai Westang Biotech Co., Ltd (Shanghai, P.R. China), Lot: WS20090520.
Isoflurane: He Bei Jiu Pai Biotech Co., Ltd, Cat: H19980141.
Acetone: Sinopharm Chemical Reagent Co., Ltd, Cat: 10000418.
Olive oil: Sinopharm Chemical Reagent Co., Ltd, Cat: 69018028.
Saline: Anhui double-crane Pharmaceutical Co., Ltd, Cat: H34023609.
Reference drug: Dexamethasone: Sigma-Aldrich, Cat: D1756-1G.
Test article and vehicle: TBD

Equipment:
Micrometer: Mitutoyo (No. 045-020, 0-25 mm, 0.001 mm, Japan)

Procedures:

Reagents setup:

Oxazolone solution: Oxazolone will be dissolved in 4:1 acetone/olive oil at 10 mg/mL

Reference drug solution: Dexamethasone will be dissolved in acetone at 2.5 mg/mL

Immunization and Challenge:

Randomize 50 animals into 5 groups (n=10).

Mice will be anesthetized with 1.0% pentobarbital sodium (60 mg/kg) and their abdomens shaved. 150 μL 3% oxazolone in 4:1 acetone/olive oil will be painted on abdomen of each mouse on day 0.

All the mice will be challenged by applying 20 μL 1% oxazolone in 4:1 acetone/olive oil onto both sides of right ear topically (10 μL/side) on day 5.

Treatment:

Compounds 1, 3 and the reference drug will be administered following different dosage protocols: a) Group 1 (vehicle group), saline will be administrated orally 1 hour before Oxazolone challenge. b) Reference drug group, dexamethasone (0.05 mg/ear) in acetone, will be applied topically (20 μL/ear, 10 μL/side) to both sides of right ear 1 hours, and 6 hours after Oxazolone challenge in Group 2. c) Different dosage of three test articles in saline will be administered orally 1 hour before Oxazolone challenge in Groups 3, 4, and 5.

Measurement:

Body weights of all mice will be recorded daily starting from Day 0 to Day 6.

Ear swelling response will be determined by ear thickness measured with a micrometer before and 24 hours after oxazolone challenge and reported as the mean change in ear thickness (ΔT±S.E.M.). Percent suppression of ear swelling response will be calculated as % suppression=[1−(ΔT of sensitized mice exposed to experimental treatment/ΔT of sensitized mice exposed to vehicle treatment)]×100.

Mice will be terminated by 95% CO₂ after the last ear thickness measurement (24 hours after Oxazolone challenge).

The ear pinnas of each group will be collected immediately after the sacrifice by punching with a 10 mm diameter punch and weighed. For group 1 both left and right ears will be collected, for group 2-5 only right ears will be collected. The ear samples will be frozen in liquid nitrogen. The sponsor will decide whether to run the MPO activity test in the other groups in 45 days after received the raw data.

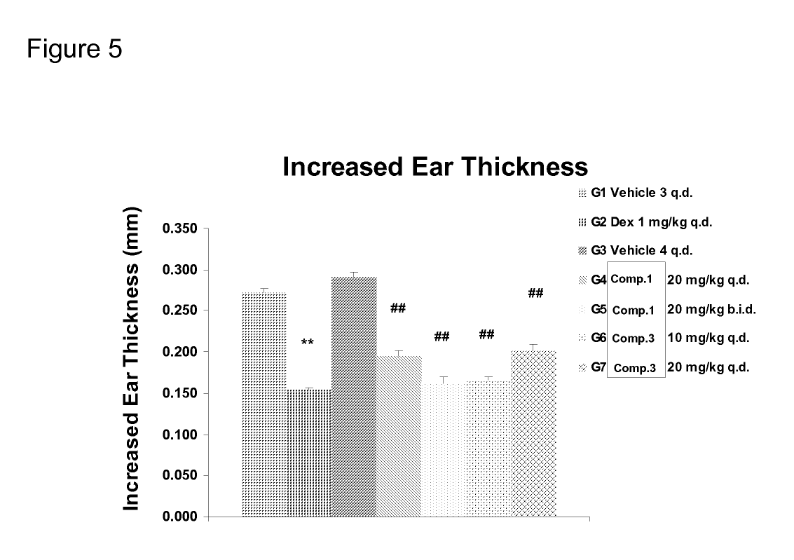

Compounds 1 and 3 have the same anti-inflammatory activity as the control (FIG. 5).

Statistics:

Results will be presented as mean±S.E.M. One-way analysis of variance followed by Dunnett's post test will be used to determine differences between groups. P<0.05 will be considered statistically significant.

### Example 8

### Use of Compound 1

A patient is suffering from an inflammatory condition. A therapeutically effective amount of Compound 1 is administered to the patient in an acceptable dosage form. It would be expected that the patient would improve his/her condition or recover.

### Example 9

### Use of Compound 2

A patient is suffering from an inflammatory condition. A therapeutically effective amount of Compound 2 is administered to the patient in an acceptable dosage form. It would be expected that the patient would improve his/her condition or recover.

### Example 10

### Use of Compound 3

A patient is suffering from an inflammatory condition. A therapeutically effective amount of Compound 3 is administered to the patient in an acceptable dosage form. It would be expected that the patient would improve his/her condition or recover.

### Example 11

### Use of Compound 1

A patient is suffering from age-related macular degeneration (AMD). A therapeutically effective amount of Compound 1 is administered to the patient in an acceptable dosage form. It would be expected that the patient would improve his/her condition or recover.

### Example 12

### Use of Compound 2

A patient is suffering from age-related macular degeneration (AMD). A therapeutically effective amount of Compound 2 is administered to the patient in an acceptable dosage form. It would be expected that the patient would improve his/her condition or recover.

### Example 13

### Use of Compound 3

A patient is suffering from age-related macular degeneration (AMD). A therapeutically effective amount of Compound 3 is administered to the patient in an acceptable dosage form. It would be expected that the patient would improve his/her condition or recover.

### Example 14

### Use of Compound 1

A patient is suffering from breast cancer. A therapeutically effective amount of Compound 11 is administered to the patient in an acceptable dosage form. It would be expected that the patient would improve his/her condition or recover.

### Example 15

### Use of Compound 2

A patient is suffering from breast cancer. A therapeutically effective amount of Compound 22 is administered to the patient in an acceptable dosage form. It would be expected that the patient would improve his/her condition or recover.

### Example 16

### Use of Compound 3

A patient is suffering from breast cancer. A therapeutically effective amount of Compound 3 is administered to the patient in an acceptable dosage form. It would be expected that the patient would improve his/her condition or recover.

Throughout this specification, various scientific publications and patents or published patent applications are referenced. The disclosure of all these patents, published applications and scientific publications in their entireties is hereby incorporated by reference in their entirety into this specification in order to more fully describe the state of the art to which the presently described subject matter pertains. Citation or identification of any reference in this section or any other part of this application shall not be construed as an admission that such reference is available as prior art to the presently described subject matter.

It will be appreciated by those skilled in the art to which the present subject matter pertains that various modifications can be made without departing from the essential nature thereof It is intended to encompass all such modification within the scope of the appended claims.

We claim:

1. A method for treating a cell migration disease, disorder or condition in a subject, comprising administering to the subject a therapeutically effective amount of a compound of general formula I 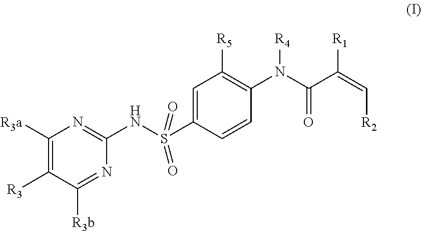
wherein
R₁ is alkyl, aryl, heteroaryl, —COR₆, —COOR₆, —NR₇R₈, —CONR₇R₈ or —NR₉COR₁₀;
R₂ is aryl or heteroaryl;
R₃, R₃a and R₃b represent H or one to three radicals selected from lower alkyl, lower alkoxy, halogen, —NR₇R₈, —COOR₆ or —CONR₇R₈ with proviso that R₃a and R₃b cannot both be H.
R₄ is H, alkyl, aryl, carbocyclyl, acyl, —O or heterocyclyl;
R₅ is H, halogen, alkyl, aryl, heteroaryl, —OR₆, —SR₆, —COR₆, —COOR₆, —NR₇R₈, —CONR₇R₈ or —NR₉COR₁₀; or R₄ and R₅ together with the carbon and nitrogen atoms to which they are attached form a 5-6 membered heterocyclic ring optionally containing a further double bond;
R₆ is H, hydrocarbyl or heterocyclyl;
R₇ and R₈ are each independently H, hydrocarbyl or heterocyclyl, or R₇ and R₈ together with the nitrogen atom to which they are attached form a 5-6 saturated heterocyclic ring, optionally containing 1 or 2 further heteroatoms selected from N, S and/or O, and wherein the further N atom is optionally substituted by lower alkyl, aralkyl, haloalkyl or hydroxyalkyl;
R₉ is H, lower alkyl or phenyl;
R₁₀ is aryl or heteroaryl,
wherein the hydrocarbyl, heterocyclyl, aryl and heteroaryl are each independently optionally substituted by one or more radicals selected from lower alkyl, halogen, aryl, heterocyclyl, heteroaryl, nitro, epoxy, epithio, —OR₆, —SR₆, —COR₆, —COOR₆, —NR₇R₈, —CONR₇R₈, —NR₇—COR₆, —SO₃R₆, —SO₂R₆, —SO₂NR₇R₈ and —NR₇SO₂R₆, wherein R₆, R₇ and R₈ are as defined above;
or an enantiomer or a pharmaceutically acceptable salt thereof.

2. A method for treating a cell migration disease, disorder or condition in a subject, comprising administering to the subject a therapeutically effective amount of a pharmaceutical composition comprising a compound of general formula I 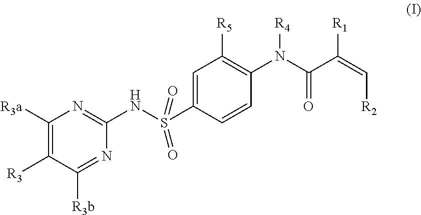
wherein
R₁ is alkyl, aryl, heteroaryl, —COR₆, —COOR₆, —NR₇R₈, —CONR₇R₈ or —NR₉COR₁₀;
R₂ is aryl or heteroaryl;
R₃, R₃a and R₃b represent H or one to three radicals selected from lower alkyl, lower alkoxy, halogen, —NR₇R₈, —COOR₆ or —CONR₇R₈ with proviso that R₃a and R₃b cannot both be H.
R₄ is H, alkyl, aryl, carbocyclyl, acyl, —O or heterocyclyl;
R₅ is H, halogen, alkyl, aryl, heteroaryl, —OR₆, —SR₆, —COR₆, —COOR₆, —NR₇R₈, —CONR₇R₈ or —NR₉COR₁₀; or R₄ and R₅ together with the carbon and nitrogen atoms to which they are attached form a 5-6 membered heterocyclic ring optionally containing a further double bond;
R₆ is H, hydrocarbyl or heterocyclyl;
R₇ and R₈ are each independently H, hydrocarbyl or heterocyclyl, or R₇ and R₈ together with the nitrogen atom to which they are attached form a 5-6 saturated heterocyclic ring, optionally containing 1 or 2 further heteroatoms selected from N, S and/or O, and wherein the further N atom is optionally substituted by lower alkyl, aralkyl, haloalkyl or hydroxyalkyl;
R₉ is H, lower alkyl or phenyl;
R₁₀ is aryl or heteroaryl,
wherein the hydrocarbyl, heterocyclyl, aryl and heteroaryl are each independently optionally substituted by one or more radicals selected from lower alkyl, halogen, aryl, heterocyclyl, heteroaryl, nitro, epoxy, epithio, —OR₆, —SR₆, —COR₆, —COOR₆, —NR₇R₈, —CONR₇R₈, —NR₇—COR₆, —SO₃R₆, —SO₂R₆, —SO₂NR₇R₈ and —NR₇SO₂R₆, wherein R₆, R₇ and R₈ are as defined above;
or an enantiomer or a pharmaceutically acceptable salt thereof.

3. The method according to claim 1, wherein the further N atom is optionally substituted by a member selected from the group consisting of pyrrolidino, piperidino, morpholino, thiomorpholino, piperazine and N-methylpiperazino.

4. The method according to claim 1, wherein
R₁ is NR₉COR₁₀;
R₂ is an optionally substituted heteroaryl;
R₃, R₃a and R₃b are H or one to three alkyl radicals; with proviso that R3a and R3b cannot both be H;
R₄ is H, alkyl, carbocyclyl, aryl, acyl, —O or heterocyclyl;
R₅ is H, halogen, alkyl, aryl, heteroaryl, —OR₆, —SR₆, —COR₆, —COOR₆, —NR₇R₈, —CONR₇R₈ or —NR₉COR₁₀; or R₄, the nitrogen atom to which it is attached and R₅ form a 5-6 membered heterocyclic ring;
R₆ is H, alkyl, aryl or heterocyclyl;
R₇ and R₈ each independently is H, alkyl, aryl or heterocyclyl, or R₇ and R₈ together with the nitrogen atom to which they are attached form a saturated 5-6 heterocyclic ring, optionally containing 1 or 2 further heteroatoms selected from N, S and/or O, and wherein said further N atom is optionally substituted by lower alkyl, optionally substituted by phenyl, halogen or hydroxy;
R₉ is H, lower alkyl or phenyl; and
R₁₀ is aryl or heteroaryl,
wherein the alkyl, carbocyclyl, heterocyclyl, aryl and heteroaryl are each independently optionally substituted by one or more radicals selected from halogen, hydrocarbyl, heterocyclyl, nitro, epoxy, epithio, OR, —SR, —COR, —COOR, —NRR′, —CONRR′, —NRCOR′, —SO₃R, —SO₂R, —SO₂NRR′ and —NRSO₂R, wherein R and R′, are each independently H, hydrocarbyl or heterocyclyl, or R and R′ together with the nitrogen atom to which they are attached form a saturated heterocyclic ring, optionally containing 1 or 2 further heteroatoms selected from N, S and/or O, and wherein the further N atom is optionally substituted by lower alkyl, aralkyl, haloalkyl or hydroxyalkyl.

5. The method of claim 4, wherein the further N atom is optionally substituted with a member selected from the group consisting of pyrrolidino, piperidino, morpholino, thiomorpholino, piperazine, and N-methylpiperazino.

6. The method according to claim 1, wherein
(i) the hydrocarbyl is a straight or branched, acyclic or cyclic, saturated, unsaturated or aromatic, hydrocarbyl radical, of 1-20 carbon atoms selected from an alkyl, alkenyl, alkynyl, carbocyclyl, aryl or an aralkyl radical;
the alkyl is a straight or branched alkyl of 1 to 10 carbon atoms (C₁-C₁₀ alkyl), optionally interrupted by one or more heteroatoms selected from O, S and/or N, and/or substituted by one or more radicals selected from the group consisting of halogen, aryl, heteroaryl, heterocyclyl, nitro, epoxy, epithio, —OR, —SR, —COR, —COOR, —NRR′, —CONRR′, —NRCOR′, —SO₃R, —SO₂R, —SO₂NRR′ and —NRSO₂R, wherein R and R′, are each independently H, hydrocarbyl or heterocyclyl, or R and R′ together with the nitrogen atom to which they are attached form a saturated 5-7 membered heterocyclic ring, optionally containing 1 or 2 further heteroatoms selected from N, S and/or O, the further N atom is optionally substituted by hydrocarbyl;
the carbocyclyl is a saturated C₅-C₆ cycloalkyl or partially unsaturated C₅-C₆ cycloalkenyl radical selected from cyclopropyl, cyclobutyl, cyclopentyl, cyclopentenyl, cyclohexyl and cyclohexenyl, optionally substituted by one or more radicals selected from the group consisting of halogen, hydrocarbyl, heterocyclyl, nitro, epoxy, epithio, OR, —SR, —COR, —COOR, —NRR′, —CONRR′, —NRCOR′, —SO₃R, —SO₂R, —SO₂NRR′ and —NRSO₂R, wherein R and R′, are each independently H, hydrocarbyl or heterocyclyl, or R and R′ together with the nitrogen atom to which they are attached form a saturated heterocyclic ring, optionally containing 1 or 2 further heteroatoms selected from N, S and/or O, and wherein the further N atom is optionally substituted by hydrocarbyl;
the aryl is a substituted or unsubstituted monocyclic, bicyclic or tricyclic aromatic carbocyclic radical of 6 to 14 carbon atoms, selected from phenyl, biphenyl, naphtyl, or anthracenyl;
(ii) the heterocyclyl is a saturated or partially unsaturated, optionally substituted, monocyclic, bicyclic or tricyclic heterocycle, of 3 to 12 ring members, of which one to three atoms is a heteroatom selected from O, S and/or N; and
(iii) the heteroaryl is a substituted or unsubstituted mono- or poly-cyclic heteroaromatic ring containing one to three heteroatoms selected from O, S and/or N.

7. The method according to claim 6, wherein
the hydrocarbyl is a straight or branched, acyclic or cyclic, saturated, unsaturated or aromatic, hydrocarbyl radical, of 1 to 10 carbon atoms; and/or
the alkyl is a C₁-C₄ alkyl selected from methyl, ethyl, n-propyl, isopropyl, sec-butyl or tert-butyl; and/or
the aryl is a substituted or unsubstituted monocyclic, bicyclic or tricyclic aromatic carbocyclic radical of 6 to 10 carbon atoms; and/or
the heterocyclyl is a saturated or partially unsaturated, optionally substituted, monocyclic, bicyclic or tricyclic heterocycle, of 5 to 10 ring members, of which one to three atoms is a heteroatom selected from O, S and/or N; and/or
the heteroaryl is selected from the group consisting of pyrrolyl, furyl, thienyl, pyrazolyl, imidazolyl, oxazolyl, thiazolyl, pyridyl, quinolinyl, isoquinolinyl, pyridazinyl, pyrimidinyl, pyrazinyl, 1,3,4-triazinyl, 1,2,3-triazinyl, 1,3,5-triazinyl, benzofuryl, isobenzofuryl, indolyl, imidazo[1,2-a]pyridyl, benzimidazolyl, benzthiazolyl, benzoxazolyl, and benzodiazepinyl.

8. The method according to claim 6, wherein
the hydrocarbyl is a straight or branched, acyclic or cyclic, saturated, unsaturated or aromatic, hydrocarbyl radical, of 1 to 6 carbon atoms; and/or
the alkyl is methyl; and/or
the heterocyclyl is a saturated or partially unsaturated, optionally substituted, monocyclic, bicyclic or tricyclic heterocycle, of 5 to 6 ring members, of which one to three atoms is a heteroatom selected from O, S and/or N; and/or
the heteroaryl is thienyl.

9. The method according to claim 6, wherein
the hydrocarbyl is a straight or branched, acyclic or cyclic, saturated, unsaturated or aromatic, hydrocarbyl radical, of 2 to 3 carbon atoms; and/or
the heterocyclyl is a member selected from the group consisting of dihydrofuryl, tetrahydrofuryl, dihydrothienyl, pyrrolydinyl, pyrrolynyl, dihydropyridyl, piperidinyl, piperazinyl, morpholino and 1,3-dioxanyl.

10. The method according to claim 1, wherein the compound of general formula I is a compound of formula Ia or Ib: 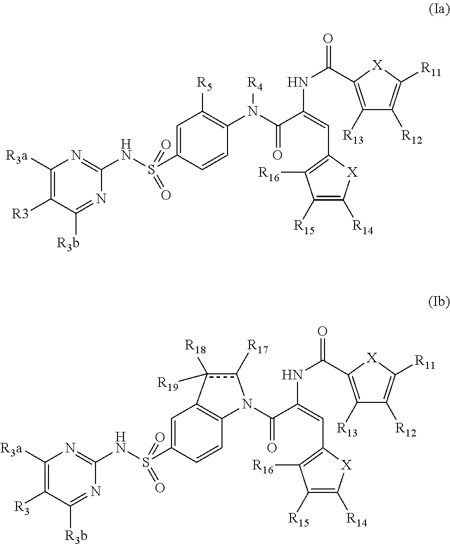
wherein
X is O, S or NH;
R₃, R₃a and R₃b are H or one to three (C₁-C₄) alkyls; with proviso that R3a and R3b cannot both be H ;
R₄ is H or (C₁-C₄) alkyl;
R₅ is H or optionally substituted (C₁-C₆) alkyl;
and R₁₁ to R₁₉, are each independently selected from H, lower alkyl, halogen, aryl, heterocyclyl, heteroaryl, nitro, epoxy, epithio, —OR₆, —SR₆, —COR_{E}, —COOR₆, —NR₇R₈, —CONR₇R₈, —NR₇—COR₆, —SO₃R₆, —SO₂R₆, —SO₂NR₇R₈ and —NR₇SO₂R₆, wherein R₆, R₇ and R₈ are each independently H, alkyl, aryl or heterocyclyl, or R₇ and R₈ together with the nitrogen atom to which they are attached form a saturated heterocyclic ring, optionally containing 1 or 2 further heteroatoms selected from N, S and/or O, and
wherein the further N atom is optionally substituted by lower alkyl, optionally substituted by phenyl, halogen or hydroxy; and
the dotted line in formula Ib represents an optional double bond.

11. The method of claim 10, wherein in the compound of formula Ia, X is S, R₃, R₃a and R₃b are H or one to three methyl groups; with proviso that R₃a and R₃b cannot both be H, R₄ is H, R₅ is H or methyl and R₁₁ to R₁₆ are H.

12. The method of claim 10, wherein the compound of formula Ia is selected from the group consisting of
compound 1 of the formula 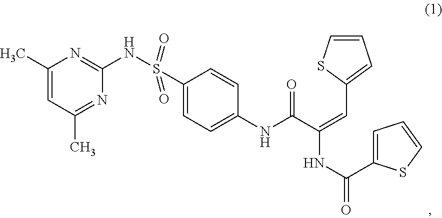
compound 2 of the formula 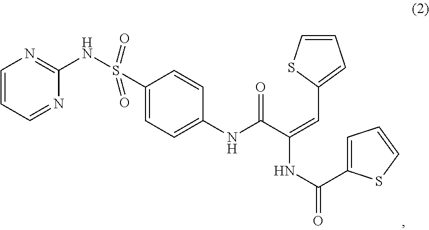
compound 3 of the formula 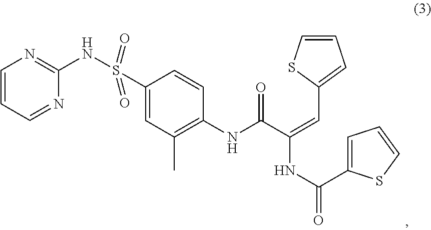
and
compound 4 of the formula

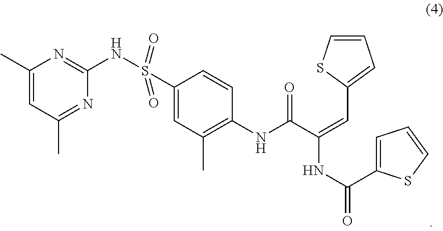

13. The method of claim 10, wherein in the compound of formula Ib, X is S, R₃, R₃a and R₃b are H or one to three methyl groups; with proviso that R₃a and R₃b cannot both be H and R₁₁ to R₁₉ are H.

14. The method of claim 10, wherein the compound of formula Ib is selected from the group consisting of
compound 5 of the formula 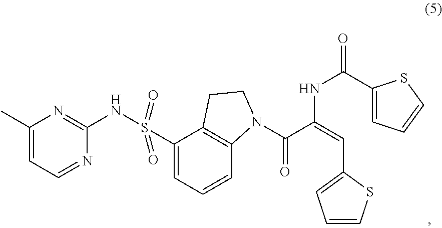
compound 6 of the formula 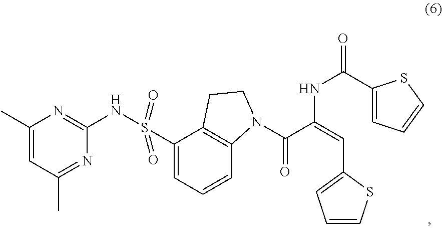
and
compound 7 of the formula

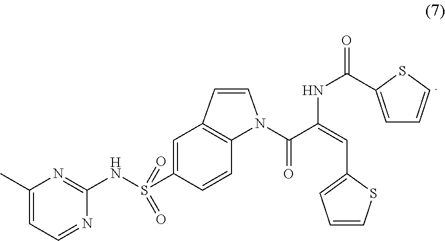

15. The method of claim 2, wherein the disease, disorder or condition is cancer.

16. The method of claim 15, wherein the cancer is selected from the group consisting of wherein the cancer is selected from the group consisting of anal cancer, astrocytoma, leukemia, lymphoma, head and neck cancer, liver cancer, testicular cancer, cervical cancer, sarcoma, hemangioma, esophageal cancer, eye cancer, laryngeal cancer, mouth cancer, mesothelioma, myeloma, oral cancer, rectal cancer, throat cancer, bladder cancer, breast cancer, uterine cancer, ovarian cancer, prostate cancer, lung cancer, colon cancer, pancreatic cancer, renal cell carcinoma, gastric cancer, skin cancer, basal cell carcinoma, melanoma, squamous cell carcinoma, oral squamous cell carcinoma, colorectal cancer, glioblastoma multiforme, endometrial cancer and malignant glioma.

17. The method of claim 15, wherein the pharmaceutical composition further comprises an effective amount of at least one anti-cancer agent.

18. The method of claim 17, wherein the at least one anti-cancer agent is selected from the group consisting of imatinib, dasatinib, axitinib, bosutinib, cediranib, erlotinib, gefitinib, lapatinib, lestaurtinib, nilotinib, semaxanib, sunitinib, toceranib, vandetanib and vatalanib.

19. The method of claim 15, further comprising administering to the subject an effective amount of at least one anti-cancer agent.

20. The method of claim 19, wherein the at least one anti-cancer agent is administered simultaneous with, before or after administration of the pharmaceutical composition.

21. The method of claim 15, wherein the pharmaceutical composition further comprises an effective amount of at least one cancer chemotherapeutic agent.

22. The method of claim 21, wherein the at least one cancer chemotherapeutic agent is selected from the group consisting of mechlorethamine, cyclophosphamide, ifosfamide, melphalan, chlorambucil, dicarbazine, streptazocine, carmustine, lomustine, semustine, chlorozotocin, busulfan, triethylenemelamine, thiotepa, hexamethylmelamine, an antimetabolite, methotrexate, fluorouracil, 5-fluorouracil, floxuridine (5′-fluoro-2′-deoxyuridine), idoxuridine, cytarabine, N-phosphonoacetyl-L-aspartate, 5-azacytidine, azaribine, 6-azauridine, pyrazofuran, 3-deazauridine, acivicin, a purine analog, thioguanine, mercaptopurine, azathioprine, pentostatin, erythrohydroxynonyladenine, a vinca alkaloid, vincristine, vinblastine, an epipodophyllotoxin, etoposide, teniposide, an antibiotic, dactinomycin, daunorubicin, doxorubicin, bleomycin sulfate, plicamycin, mitomycin, an enzyme, L-asparaginase, a platinum coordination complex, cisplatin, carboplatin, hydroxyurea, procarbazine, mitotane, a hormone, an adrenocorticosteroid, prednisone, prednisolone, aminoglutethimide, a progestin, hydroxyprogesterone caproate, medroxyprogesterone acetate, megesterol acetate, estrogen, an androgen, diethylstilbestrol, fluoxymesterone, ethynyl estradiol, an antiestrogen, tamoxifen, a gonadotropin-releasing hormone analog and leuprolide.

23. The method of claim 15, further comprising administering to the subject an effective amount of at least one cancer chemotherapeutic agent.

24. The method of claim 23, wherein the at least one cancer chemotherapeutic agent is administered simultaneous with, before or after administration of the pharmaceutical composition.

25. The method of claim 15, further comprising administering to the subject radiation therapy.

26. The method of claim 25, wherein radiation therapy is administered simultaneous with, before or after administration of the pharmaceutical composition.

27. The method of claim 2, wherein the disease, disorder or condition is an inflammatory condition.

28. The method of claim 27, wherein the inflammatory condition is selected from the group consisting of pulmonary fibrosis, ischaemic heart disease, Crohn's disease, dermatomyositis, diabetes mellitus, Guillain-Barre syndrome, hashimoto's disease, idiopathic thrombocytopenic purpura, mixed connective tissue disease, myasthenia gravis, narcolepsy, pemphigus vulgaris, pernicious anaemia, polymyositis, primary biliary cirrhosis, Sjogren's syndrome, temporal arteritis, ulcerative colitis, vasculitis, Wegener's granulomatosis, systemic lupus erythematosus, lupus nephritis, Goodpasture's syndrome, haemolytic anaemia, thyrotoxicosis, multiple sclerosis, scleroderma, asthma, rheumatoid arthritis, osteoarthritis, septicaemia, artherosclerosis, chronic renal disease, inflammatory bowel disease, vasculitis, peritonitis, giant papillary conjunctivitis, uveitis, seasonal allergic conjunctivitis, chronic prostatitis, glomerulonephritis, hypersensitivities, inflammatory bowel diseases, pelvic inflammatory disease, reperfusion injury, transplant rejection, Chediak-Higashi syndrome, chronic granulomatous disease, urinary tract inflammatory conditions, interstitial cystitis, ulcerative colitis, systemic sclerosis, dermatomyositis, polymyositis and inclusion body myositis.

29. The method of claim 27, wherein the pharmaceutical composition further comprises at least one anti-inflammatory agent.

30. The method of claim 29, wherein the anti-inflammatory agent is selected from the group consisting of a corticosteroid, cortisol, aldosterone, hydrocortisone, hydrocortisone acetate, cortisone acetate, tixocortol pivalate, prednisolone, methylprednisolone, prednisone, triamcinolone acetonide, triamcinolone alcohol, mometasone, amcinonide, budesonide, desonide, flucinonide, fluocinolone acetonide, halcinonide, betamethasone, betamethasone sodium phosphate, dexamethasone, dexamethasone dodium phosphate, flucortolone, hydrocortisone-17-butyrate, hydrocortisone-17-valerate, aclometasone dipropionate, betamethasone valerate, betamethasone dipropionate, prednicarbate, clobetasone-17-butyrate, clobetasol-17-propionate, flucortolone caproate, fluocortolone pivalate, fluprednidene acetate, a non-steroidal anti-inflammatory, a cox-2 inhibitor, nimesulide, diclofenac, licofelone, aspirin, ibuprofen, naproxen, an immune selective anti-inflammatory derivative, phenylalanine-glutamine-glycine, an herb, Harpagophytum, hyssop, ginger, turmeric, Arnica Montana, willow bark and cannabis.

31. The method of claim 27, further comprising administering to the subject an effective amount of at least one anti-inflammatory agent.

32. The method of claim 31, wherein the at least one anti-inflammatory agent is administered simultaneous with, before or after administration of the pharmaceutical composition.

33. The method of claim 2, wherein the cell migration disease, disorder, or condition is an excessive angiogenesis condition.

34. The method of claim 33, wherein the excessive angiogenesis condition is selected from the group consisting of diabetic blindness, chronic inflammation, arthritis, age-related macular degeneration, retinopathy, rheumatoid arthritis, osteoarthritis, Crohn's disease, psoriasis, cancer, Alzheimer's disease, restenosis, pulmonary fibrosis, asthma, angiofibroma, neovascular glaucoma, arteriovenous malformations, nonunion fractures, lupus, a connective tissue disorder, Osler-Weber syndrome, atherosclerotic plaque, corneal graft neovascularization, pyogenic granuloma, retrolental fibroplasias, scleroderma, granulation, hemangioma, trachoma, hemophilic joints, peritoneal endometriosis, adiposity and vascular adhesions.

35. The method of claim 33, wherein the pharmaceutical composition further comprises at least one angiogenesis inhibitor.

36. The method of claim 35, wherein the at least one angiogenesis inhibitor is selected from the group consisting of bevacizumab, sunitinib, sorafenib, thalidomide, lenalidomide, panitumumab, cetuximab and erlotinib.

37. The method of claim 2, further comprising administering to the subject a therapeutically effective amount of at least one member selected from the group consisting of an anti-cancer agent, an angiogenesis inhibitor and an anti-inflammatory agent.

38. The method of claim 2, wherein the pharmaceutical composition further comprises an effective amount of at least one member selected from the group consisting of an anti-cancer agent, an angiogenesis inhibitor and an anti-inflammatory agent.

39. The method of claim 15, wherein the pharmaceutical composition further comprises an anti-cancer agent, an angiogenesis inhibitor and optionally an anti-inflammatory agent.
